(19) United States
(12) Patent Application Publication
(10) Pub. No.: US 2006/0029958 A1
(43) Pub. Date: Feb. 9, 2006

(54) Method for the identification and isolation of strong bacterial promoters

(76) Inventors: Vehary Sakanyan, Orvault (FR); Mikael Dekhtyar, Tver (RU); Amelie Morin, Nantes (FR); Frederique Braun, Nantes (FR); Larissa Modina, Nantes (FR)

Correspondence Address: YOUNG & THOMPSON, 745 SOUTH 23RD STREET, 2ND FLOOR, ARLINGTON, VA, 22202, US

(21) Appl. No.: 11/189,731

(22) Filed: Jul. 27, 2005

Related U.S. Application Data
(63) Continuation of application No. PCT/EP04/01742, filed on Jan. 23, 2004.

(30) Foreign Application Priority Data
Jan. 27, 2003 (EP) 03290203.3

(51) Int. Cl. C12Q 1/68; G06F 19/00; G01N 33/48; G01N 33/50

(52) U.S. Cl. 435/6; 702/20

(57) ABSTRACT

The present invention relates to the identification and the isolation from bacterial genomes of new sequences having strong bacterial promoter activity. The invention also concerns new nucleic acids having strong bacterial promoter activity and their uses for improving RNA and/or protein synthesis using cellular (in vivo) or cell-free (in vitro) expression systems.

This application is a continuation of PCT/EP2004/001742, filed Jan. 23, 2004, which designated the United States and claims priority of European application No. 03290203.3, filed Jan. 27, 2003, the entire contents of each of the above-identified applications are hereby incorporated by reference.

The present invention relates to the identification and the isolation from bacterial genomes of new sequences having strong bacterial promoter activity. The invention also concerns new nucleic acids having strong bacterial promoter activity and their uses for improving RNA and/or protein synthesis using cellular (in vivo) or cell-free (in vitro) expression systems.

Recombinant protein production in bacterial cells is a major area of biotechnology. Examples of recombinant molecules of interests synthesized in bacteria are antigens, antibodies and fragments thereof for vaccines, enzymes in medicine or agro-food industry, hormones, cytokines or growth factor in medicine or agronomy.

High throughput technologies and in particular protein array methods for analyzing protein-molecules interactions (EP 01402050.7), needs also to provide protein or polypeptide of interest, such as an antigen, an antibody, a receptor for identifying ligands, agonists or antagonists thereof.

Synthesis of a desired mRNA can also be convenient for their subsequent use in protein synthesis, in diagnosis or in anti-sense therapeutic approach for example.

Many microbial overexpression systems have been developed to achieve high yield of protein synthesis.

Usual methods of recombinant protein synthesis include in vivo expression of recombinant genes from strong promoters in corresponding host cells, such as bacteria, yeast or mammalian cells or in vitro expression from a DNA template in cell-free extracts, such as the S30 system-based method developed by Zubay (1973), the rabbit reticulocyte system-based method (Pelham and Jackson, 1976) or wheat germ lysate system-based method (Roberts and Paterson, 1973). Cell-free synthesis has been applied for polysome display screening antibodies (Mattheakis et al., 1996), truncation test (van Essen et al., 1997), scanning saturation mutagenesis (Chen et al., 1999), site-specific incorporation of unnatural amino acids into proteins (Thorson et al., 1998), stable-isotope labeling of proteins (Kigawa et al., 1999) and protein array screening molecular interactions (EP 01402050.7).

The best known, expression systems in the art are based on the use of strong transcriptional signals. As an example, strong phage promoters are widely used for gene expression and protein production both in living cells or cell-free extracts.

However, improvements at the different steps of gene expression are still required to increase the yield of RNA or protein synthesis in an expression system as well as to improve the performance of overexpression of a given protein. If the different components involved in transcription are well-known in the Art, the specific contribution of each component is still controversial.

Transcription initiation can be considered as one of the rate-limiting step in mRNA synthesis, thereby for protein synthesis as well. Therefore, identification and use of strong promoters in microbial genomes can lead to the development of new in viva and in vitro protein overexpression systems. Furthermore, studying strong promoters is important for the elucidation of a global transcriptional regulation of highly expressed genes and operons in the context of a whole organism and further improving the performance of protein overexpression in cellular as well as in cell-free systems.

RNA polymerase is a unique enzyme required for transcription of genes in all bacteria. Its core-enzyme consists of subunits α (in a dimeric state) β′, β and ω, which binds exchangeable σ subunits and forms a holoenzyme able to recognize a promoter sequence and to initiate transcription. The assemblage of a core enzyme occurs in the following order α→α2→α2β→α2ββ′ (Kimura et Ishihama, 1996). In a majority of promoters, consensus sequences TATAAT (site −10) et TTGACA (site −35) determine the recognition of a major c subunit considered as an analogue of Escherichia coli σ⁷⁰ factor.

The strength of a major σ-dependent bacterial promoters is determined by a rate of homology of their −10 et −35 sites with corresponding consensus sequences and by the length of a distance (spacer) between these sites that should be 17±1 bp. However, the strong promoter recognition depends also on binding RNA polymerase α subunit to a 17-20 bp AT-rich sequence located just upstream the −35 site and known as a UP-element (Ross et al., 1993). A consensus sequence 5′NNAAAWWTWTTTTNNNAAANNN (where W is A or T and N is any of four bases) was established for E. coli UP element by sequence analysis of artificially created sequences providing high gene expression (Estrem et al., 1998). This consensus can be divided into two parts, a proximal AAAAAARNR (where R is A or G) and a distal subsite NNAWWWWWTTTTTN (Estrem et al., 1999). Searching for similar sequences located upstream of previously detected promoters in the E. coli genome (Thieffry et al., 1998; http://www.cifn.unam.mx/Computational Biology/E.coli-predictions) with a software GCG version 9.0 allowed to detect 32 putative promoters having ≦4 mismatches in the full UP element consensus (Estrem et al., 1999). Extended AT-rich sequences, which can be considered as UP elements or UP element-like sequences have been also detected in bacteria Clostridium pasterianum (Graves et al., 1986), Bacillus subtilis (Fredrick et al., 1995), Bacillus stearothermophilus (Savchenko et al., 1998) and Vibrio natrigens (Aiyar et al., 2002). The presence of such a sequence in a promoter can rise up to 330-fold gene expression in Escherichia coli cells (Aiyar et al., 1998). The N-terminal domain of α subunit is responsible for assemblage of RNA polymerase whereas the C-terminal domain is implicated into contacts to UP-element and other transription activators (Ross et al., 2001).

Thus, a UP-element of strong promoters seems to play an essential role In the modulation of the level of mRNA synthesis in bacterial cells.

Consequently, in the present invention, it has been further confirmed that the α subunit of RNA polymerase plays a determinant role in increasing RNA and protein synthesis in cell-free systems, as compared to the other subunits of a core-enzyme of RNA polymerase.

As used herein, a “cellular system for in vivo RNA or protein synthesis” refers to a system enabling RNA or protein synthesis including a host cell comprising an appropriate recombinant DNA template for the expression of a gene of interest and subsequent synthesis of RNA or protein of interest

As used herein, a “cell-free system” or “cell-free synthesis system refers to any system enabling the synthesis of a desired protein or of a desired RNA from a DNA template using cell-free extracts, namely cellular extracts which do not contain viable cells. Hence, it can refer either to in vitro transcription-translation or in vitro translation systems. Examples of eucaryotic in vitro translation methods are based on the extracts obtained from rabbit reticulocytes (Pelham and Jackson, 1976), or from wheat germ cells (Roberts and Paterson, 1973). The E. coli S30 extract-based method described by Zubay (1973) is an example of a widely used prokaryotic in vitro translation method.

The term “protein” refers to any amino-acid sequence.

The inventors have now developed new tools for the identification of nucleic acid sequences carrying putative strong bacterial promoter. The inventors have also isolated nucleic acid sequences having strong bacterial promoter activity.

As used herein the term “nucleic acid” or “nucleic acid sequence” includes RNA, DNA fragment, polynucleotide or oligonucleotide, cDNA, genomic DNA and messenger RNA.

For suitable reading of the present text, the chemical structure of a nucleic acid will be characterized by a nucleotide sequence represented by a chain of “A”, “G”, “C” or “T”, as usual for the one skilled in the Art. Of course, when a sequence is given for a double-strand DNA, it implicitely means that the reverse complementary sequence forms the other strand of such DNA.

The term “promoter” or “promoter activity” is used in the present text to refer to the capacity of a nucleic acid when inserted immediately upstream an Open Reading Frame or a sequence coding for tRNA or rRNA to promote transcription of said sequences.

Method for measuring promoter activity are well-known in the Art. The promoter activity can be measured for example according to the method below:
The nucleic acid whose promoter activity is measured, is placed immediately upstream an Open Reading Frame of a reporter gene,
The resulting construction is placed in an appropriate vector and introduced into E. coli cells,
The E. coli cells are cultured in conditions appropriate for expression of the reporter gene,
Transcriptional expression of the reporter gene is determined and compared with the transcriptional expression of the same reporter gene placed downstream a control promoter.

Instead of determining transcriptional expression, it is also possible to determine protein synthesis of a reporter protein, since transcriptional activation is usually the rate limiting step for protein synthesis. A specific method for measuring the promoter activity of a nucleic acid in a cell-free system by determining protein synthesis of ArgC reporter protein is described in the example.

According to the present invention, a nucleic acid is considered to have a strong bacterial promoter activity when transcriptional expression of a gene inserted downstream said nucleic acid is higher than the transcriptional expression of the same gene inserted downstream a control bacterial strong promoter, such as the ptac promoter.

A first object of the invention is a method for the identification of a nucleic acid sequence carrying a putative bacterial strong promoter, said method comprising:
a. selecting among the sequences of a nucleic acid database, a putative promoter sequence of at least 50 nucleotides, preferably around 60-70 nucleotides, said putative promoter sequence being located upstream the initiation codon of an Open Reading Frame or a sequence corresponding to tRNA or rRNA, in a region which does not extend further than 500 nucleotides, preferably 300 nucleotides from said initiation codon, said putative promoter sequence comprising an UP element, said UP element consisting of either
   the following consensus pattern: AAAWWTWTTTTNNNAAA (SEQ ID NO:1), wherein “W” stands for any of the symbols “A” or “T” and “N” stands for any of the four symbols “A”, “T”, “G” or “C”; or,
   a nucleotide sequence of the same length of SEQ ID NO:1 which can be aligned with SEQ ID NO:1 and having a score similarity sUP which is equal or superior to a minimal score similarity parameter scUP,
b. selecting among the sequences selected in step a., the sequences comprising a −35 site located from 0 to 5 nucleotides downstream the UP element, said −35 site consisting of either
   the following consensus pattern TCTTGACAT (SEQ ID NO:2), or
   a nucleotide sequence of the same length of SEQ ID NO:2 which can be aligned with SEQ ID NO:2 and having a score similarity s35 which is equal or superior to a minimal score similarity parameter sc35; and
c. identifying among the sequences selected in step b., a sequence comprising a −10 site, downstream the −35 site, preferably at a distance from 14 to 20 nucleotides, preferably from 15 to 19, better from 16 to 18, and optimally 17 nucleotides from the −35 site, said −10 site consisting of either
   the following consensus pattern TATAAT (SEQ ID NO:3), or
   a nucleotide sequence of the same length of SEQ ID NO:3 which can be aligned with SEQ ID NO:3 and having a score similarity s10 which is equal or superior to a minimal score similarity parameter sc10.

As used herein, the term “putative strong promoter” means that there is a high probability the sequence carry a strong promoter.

As used herein, the term “nucleic acid database” means a database which gathers sequence information obtained by the sequencing of nucleic acids. Especially, the database gathers genomic sequences information. Databases from micro-organism genomes such as prokaryotes are especially preferred.

In a preferred embodiment, searched nucleic acid databases are selected among the genome having a percentage of adenine and thymine inferior to 65%, more preferably, inferior to 50%. Indeed, it has been shown that these databases enable the identification of a high number of strong promoters.

Preferably, the nucleic acid databases comprise genomic sequences from bacterial species from bacteria which is used in the industry and whose genome comprises a percentage of adenine and thymine inferior to 65%.

Examples of such bacterias are listed in Table 5.

Particularly are preferred bacterial nucleic acid databases comprising genomic sequences from one bacterial specie selected from the group consisting of Thermatoga maritima, Mycobacterium tuberculosis, Mycobacterium leprae, Pseudomonas aeruginosa, Brucella melitensis, Neisseria meningitis, Salmonella typhimurium, Escherichia col, Vibrio cholerae, Yersinia pestis, Streptococcus pneumoniae, Streptococcus pyogenes, Haemophilus influenzae and Helicobacter pylori.

One example of the present invention is the use of the method for identifying nucleic acid sequence from bacterial nucleic acid database of T. maritima genomic sequences.

The similarity scores between two aligned sequences referred by sUP, s35 and s10 correspond to the sum of each coincidence rates of symbols in the corresponding alignments: the identity rate is equal to 1, the non-identity rate is 0.5 or 0 and is determined for each pair of compared symbols as follows:
0.5 for pairs “A” to “T” or
   “T” to “A” and
0 for other possible pairs.

Therefore, the similarity score between each consensus pattern and the aligned sequence varies from 0 to the corresponding length of the pattern, namely 17 for UP element, 9 for −35 site and 6 for −10 site.

The minimal acceptable value for sUP, s35 and s10 for selecting the putative promoter are defined by the parameters scUP, sc35 and sc10 which can be determined empirically depending upon the nature of the database, the size of the database, the number and the strength of promoters to be identified by the method.

In a preferred embodiment of the method, scUP is at least equal to 11, sc35 is at least equal to 5, and sc10 is at least equal to 4. Such combination of parameters for minimal score similarity are particularly preferred for the screening of databases of Thermotoga maritima genomic sequences.

In a particular embodiment of the method, a normalised score is attributed to each identified sequence enabling the comparison of the putative strength for each identified sequence.

According to one specific embodiment of the method, a normalised score tot_sc is attributed to each identified sequence according to the following equation:
tot_{—}sc=0.30*[1−(17−sUP)/20]+0.25°[1−(9−s35)/10]+0.25°[1−(6−s10)²/10]+0.2*nsc_{—}dist, wherein nsc_dist is defined according to the following table 1:

| | | | | | |
|---|---|---|---|---|---|
| Distance between | 17 | 16, 18 | 15, 19 | 14, 20 | Other |
| −35 site and −10 site | | | | | |
| in nucleotides | | | | | |
| nsc_dist | 1 | 0.95 | 0.85 | 0.7 | 0.2 |

and the method further comprises the step of selecting the sequences having a normalised score tot_sc superior to 0.85.

Of course, any other methods of calculation of the normalised score which enable similar comparison of the strength of the identified promoters can be applied.

The formula of the normalized score should reflect the inexact matching for the different subregions, e.g., the UP element, the −35 site and the −10 site and the relative importance of corresponding subregions and the spacer for the evaluation of the promoter strength. The rate of similarity for each subregion can be modulated by increasing or decreasing the attached coefficients. However, it has been shown that the set of sequences having strong promoter activity identified by the method of the invention does not essentially depend upon small variation of the coefficients.

Indeed, the inventors have shown that a majority of promoters identified from T. maritima genome and having a score superior to 0.85 according to the above defined equation, have strong promoter activity.

Naturally, the invention also relates to a computer program comprising computer program code means for instructing a computer to perform the method of the invention.

The invention further concerns a computer readable storage medium having stored therein a computer program according to the invention.

Another aspect of the invention is a method for the isolation of a nucleic acid having strong bacterial promoter activity, wherein said method further comprises the steps of:
a. isolating a nucleic acid having a putative strong bacterial promoter, said nucleic acid sequence being identified according to the method defined above,
b. determining promoter activity of the isolated nucleic acid as compared to a control bacterial strong promoter, such as the ptac promoter,
wherein a higher promoter activity than the promoter activity of the control strong promoter indicates that said isolated nucleic acid has a strong bacterial promoter activity.

Any appropriate means for determining promoter activity of said isolated nucleic acid can be used for the method of the invention. A preferred method is described in an example as the detection of synthesis of the reporter protein ArgC in a cell-free system. Obviously, other reporter protein can also be used.

By implementing the method of the invention, the inventors have identified new nucleic acids derived from T. maritima genomic sequence, having a strong bacterial promoter activity.

Another aspect of the invention thus relates to an isolated nucleic acid having a strong bacterial promoter activity, characterized in that it is obtainable by the method defined above and in that it consists of
a. a nucleic acid sequence selected among the group consisting of SEQ; ID NOs 4-16;
b. a modified nucleic acid sequence having at least 70%, preferably at least 80%, and better at least 90% identity when aligned with one of SEQ ID NOs 4-16,
c. a modified nucleic acid sequence which hybridizes under stringent conditions with one of the sequences of SEQ ID NOs 4-16, or,
d. a nucleic acid sequence comprising the following consensus pattern; GNAAAAAtWTNTTNAAAAAAMNCTTGAMA(N)₁₈TATAAT (SEQ ID NO:21) wherein “W” stands for any of the symbols “A” or “T”, “N” stands for any of the four symbols “A”, T, “G” or “C” and “M” stands for “A” or “C”, wherein said modified nucleic acid is between 50 and 300 nucleotides long, preferably between 50 and 100 nucleotides long, and retains substantially the same promoter activity as the non-modified sequence to which it can be aligned.

The nucleic acid of SEQ ID NOs 4-16 are more specifically defined in FIG. 1 and in example 2.

For evaluating the similarity of a modified sequence with one of SEQ ID Nos 4-16, the alignment program BLASTA (Altschul et al., 1990) is used.

As used herein, the term <<stringent conditions>> refers to the conditions enabling specific hybridisation of the single strand nucleic acid at 65° C. for example in a solution consisting of 6× SSC, 0.5% SDS, 5× Denhardt's solution and 100 mg of non specific DNA carrier, or any other solution of the same ionic strength, and after a washing at 65° C., for example in a solution consisting of 0.2× SSC and 0.1 SDS or any other solution of the same ionic strength. The parameters which define the stringency conditions are the temperature at which 50% of the stands are separated (Tm). For nucleic acids more than 30 bases, Tm is defined as follows: Tm=81.5+0.41 (% G+C)+16.6 Log (concentration in cations)−0.63 (% formamide)−(600/number of bases). Stringency conditions can be adapted according to the size of the sequence and the content of GC and all other parameters, according to the protocols described in Sambrook et al.

Modified nucleic acids derived from SEQ ID NOs 4-16 which retains substantially the same promoter activity as the non-modified from which it can be aligned are also concerned by the present invention.

According to the invention, it will be considered that a modified sequence retains substantially the same promoter activity as the non modified sequence from which it can be aligned if the measured promoter activity is not inferior to 70%, preferably 80%, and more preferably 90% than that of the non-modified sequence to which it can be aligned.

Of course, modified sequence having a higher promoter activity than the non-modified sequence from which it can be aligned are comprised in the present invention.

Preferably, such modified sequence is a sequence which has been modified by deletion or mutagenesis. Preferred modifications are nucleotides substitutions which do not fall in the regions comprising the UP element, the −35 site and the −10 site as defined above. Other preferred modifications are nucleotide substitutions which increase similarity of the UP element, −35 site or the −10 site with the corresponding consensus pattern as defined above. Another preferred modification is a modification of the length of the distance separating the −35 and the −10 site to render it closer to the optimal distance of 17±1 nucleotides.

Naturally, such preferred modifications would not necessarily increase the strength of the promoter, but the one skilled in the Art can screen the promoter activity of the modified sequence, in order to select the appropriate modifications.

The nucleic acid having strong bacterial promoter activity are more specifically useful for the synthesis of a protein and/or RNA of interest.

Another aspect of the invention is thus an expression cassette comprising a nucleic acid having strong bacterial promoter activity according to the invention.

As used herein, an expression cassette is a means for inserting into, a sequence encoding a protein of interest and for synthesizing said protein into a host cell or in a cell-free system.

The expression cassette preferably is a DNA molecule containing a multiple cloning site immediately downstream the nucleic acid having strong bacterial promoter activity of the invention. The multiple cloning site enables the insertion using restriction enzymes and ligase of the sequence encoding the protein of interest.

Preferably, the expression cassette is characterized in that it is a plasmid, a cosmid or a phagemid for in vivo protein synthesis.

Advantageously, the expression cassette of the invention further comprises an Open Reading Frame encoding α subunit of a RNA polymerase under the control of a promoter appropriate for expression in said host cell.

The invention also relates to a DNA template for RNA or protein synthesis, comprising the nucleic acid having strong bacterial promoter activity of the invention, inserted upstream an Open Reading Frame encoding a protein of interest.

According to the invention, a “protein of interest” refers to any type of protein characterised in that it is not naturally expressed from the nucleic acid having strong bacterial promoter activity of the invention.

Examples of protein of interest are enzymes, enzyme regulators, receptor ligands, haptens, antigens, antibodies and fragments thereof.

In order to simplify the reading of the present text, as used herein, the term “DNA template” refers to a nucleic acid comprising the following elements:
an Open Reading Frame with an initiation codon and a stop codon encoding a protein of interest;
the nucleic acid having strong bacterial promoter activity as here-aboved defined, located upstream the Open Reading Frame encoding a protein of interest;
optionally specific signals for translation initiation and termination;
optionally, specific signals for transcription termination;
optionally, specific signals for binding transcriptional activating proteins;
optionally, a sequence in frame with said Open Reading Frame, encoding a tag for convenient purification or detection.

The selection of the different above-mentioned elements depends upon the selected expression system.

Preferably, the nucleic acid having strong bacterial promoter activity of the invention is located immediately upstream the initiation codon of the Open Reading Frame encoding the protein of interest

In cell-free systems, linear DNA templates may affect the yield of RNA or protein synthesis and their homogeneity because of nuclease activity in the cell-free extract. By “protein homogeneity”, it is meant that a major fraction of the synthesized product correspond to the complete translation of the Open Reading Frame, leading to full-length protein synthesis and only a minor fraction of the synthesized proteins correspond to interrupted translation of the Open Reading Frame, leading to truncated forms of the protein. Thus, the desired protein synthesis is less accompanied by truncated polypeptides.

The use of elongated DNA template according to the invention, improves the yield and the homogeneity of synthesized proteins in cell-free systems.

Thus, in a preferred embodiment, a linear DNA template further comprises an additional DNA fragment, which is at least 3 bp long, preferably longer than 100 bp, and more preferably longer than 200 bp, located immediately downstream the stop codon of the Open Reading Frame encoding the desired RNA or protein of interest.

It has also been shown that the use of DNA template further comprising an additional DNA fragment containing transcriptional terminators, improves the yield and the homogeneity of the protein synthesis from cell-free systems.

One example of transcription terminators which can be used in the present invention is the T7 phage transcriptional terminator.

The DNA template of the invention are useful in a method for RNA or protein synthesis from a DNA template comprising the steps of
a. providing a cellular or cell-free system enabling RNA or protein synthesis from the DNA template according to the invention;
b. recovering said synthesized RNA or protein.

The strong bacterial promoter contained in the used DNA template are particularly efficient to bind α subunit of RNA polymerase.

In a preferred embodiment, in order to increase the yield of RNA and/or protein synthesis, the concentration of α subunit of RNA polymerase, but not of other subunits, is increased in said cellular or cell-free system, comparing to is natural concentration.

As used herein, the term “natural concentration” refers to the concentration of the RNA polymerase α subunit established in vivo in bacterial cells without affecting the growth conditions or the concentration of the RNA polymerase α subunit in vivo reconstituted holoenzyme from purified subunits.

The increase of the concentration of the α subunit can refer, either to an increase of the concentration of an α subunit which is identical to the one initially present in the selected expression system, or to an α subunit which is different but which can associate with β,β and ω subunits in initially present in the expression system to form the holoenzyme. For example, said different α subunit can be a mutated form of the α subunit, initially present in the selected expression system or a similar form from a related organism, provided that the essential αCTD and/or αNTD domains are still conserved or a chimaeric from related organisms.

The α subunit used is, for example, obtained from E. coli or T. maritime.

Preferably, the α subunit is derived from the same organism as the one from which is derived the strong promoter used in the DNA template and which can be obtained by the method of the invention.

In one specific embodiment, said system enabling RNA or protein synthesis from the DNA template of the invention is a cellular system.

The DNA templates can be adapted for any cellular system known in the Art. The one skilled in the Art will select the cellular system depending upon the type of RNA or protein to synthesize.

In one aspect of the invention, a cellular system comprises the culture of prokaryotic host cells. Preferred prokaryotic host cells include Streptococci, Staphylococci, Streptomyces and more preferably, B. subtilis or E. coli cells.

In a preferred embodiment, a host cell selected for the cellular expression system is a bacteria, preferably an Escherichia coli cell.

Host cells may be genetically modified for optimising recombinant RNA or protein synthesis. Genetic modifications that have been shown to be useful for in vivo expression of RNA or protein are those that eliminate endonuclease activity, and/or that eliminate protease activity, and/or that optimise the codon bias with respect to the amino acid sequence to synthesize, and/or that improve the solubility of proteins, or that prevent misfolding of proteins. These genetic modifications can be mutations or insertions of recombinant DNA in the chromosomal DNA or extra-chromosomal recombinant DNA. For example, said genetically modified host cells may have additional genes, which encode specific transcription factors interacting with the promoter of the gene encoding the RNA or protein to synthesize.

Prior to introduction into a host cell, the DNA template is incorporated into a vector appropriate for introduction and replication in the host cell. Such vectors include, among others, chromosomic vectors or episomal vectors or virus-derived vectors, especially, vectors derived from bacterial plasmids, phages, transposons, yeast plasmids and yeast chromosomes, viruses such as baculoviruses, papoviruses and SV40, adenoviruses, retroviruses and vectors derived from combinations thereof, in particular phagemids and cosmids.

For enabling secretion of translated proteins in the periplasmic space of gram bacteria or in the extracellular environment of cells, the vector may further comprise sequences encoding secretion signal appropriate for the expressed polypeptide.

The selection of the vector is guided by the type of host cells which is used for RNA or protein synthesis.

One preferred vector is a vector appropriate for expression in E. coli, and more particularly a plasmid containing at least one E. coli replication origin and a selection gene of Resistance to an antibiotic, such as the Ap^{R} (or bla) gene.

In one embodiment, the cellular concentration of α subunit of RNA polymerase is increased by overexpressing in the host cell, a gene encoding an α subunit of RNA polymerase.

Preferably, a gene encoding an α subunit of RNA polymerase is a gene form E. coli, T. maritima, T. neapolitana or T. thermophilus.

For example, the host cell can comprise, integrated in the genome, an expression cassette comprising a gene encoding an α subunit of RNA polymerase under the control of an inducible or derepressible promoter, while the expression of the other subunits remains unchanged.

An expression cassette comprising a gene encoding an α subunit of RNA polymerase can also be incorporated into the expression vector comprising the DNA template of the invention, or into a second expression vector.

For example, the expression cassette comprises the E. coli gene rpoA, under the control of a T7 phage promoter.

In a preferred embodiment, the concentration of α subunit in a cellular system is increased by induction of the expression of an additional copy of the gene encoding α subunit of RNA polymerase while expression of the other subunits remains unchanged.

In another specific and preferred use of said DNA template of the invention, said system enabling RNA or polypeptide synthesis from the DNA template according to the invention, is a cell-free system comprising a bacterial cell-free extract.

For cell-free synthesis, the DNA template can be linear or circular, and generally includes the sequence of the Open Reading Frame corresponding to the RNA or protein of interest and sequences for transcription and translation initiation. Lesley et al., (1991) optimised the Zubay (1973) E. coli S30 based-method for use with PCR-produced fragments and other linear DNA templates by preparing a bacterial extract from a nuclease-deficient strain of E. coli. Also, improvement of the method has been described by Kigawa et al. (1999) for semi-continuous cell-free production of proteins.

When a cell-free extract is used for carrying out the method of the invention, the concentration of α subunit of RNA polymerase is preferably increased by adding purified α subunit of RNA polymerase to the cell free extract. When using the DNA templates of the invention, it is indeed preferred that no other subunits of RNA polymerase are added to the cell-free extract, so that the stoechiometric ratio of α subunit/other subunits is increased in the cell-free extract in favour to the α subunit. Preferably, said purified α subunit is added in a cell-free extract, more preferably a bacterial cell-free extract, to a final concentration comprised between 15 μg/ml and 200 μg/ml.

Purified α subunit of RNA polymerase can be obtained by the expression in cells of a gene encoding an α subunit of RNA polymerase and subsequent purification of the protein. For example, α subunit of RNA polymerase can be obtained by the expression of the rpoA gene fused in frame with a tag sequence in E. coli host cells, said fusion enabling convenient subsequent purification by chromatography affinity.

The term “bacterial cell-free extract” as used herein defines any reaction mixture comprising the components of transcription and/or translation bacterial machineries. Such components are sufficient for enabling transcription from a deoxyribonucleic acid to synthesize a specific ribonucleic acid, i.e mRNA synthesis. Optionally, the cell-free extract comprises components which further allow translation of the ribonucleic acid encoding a desired polypeptide, i.e polypeptide synthesis.

Typically, the components necessary for mRNA synthesis and/or protein synthesis in a bacterial cell-free extract include RNA polymerase holoenzyme, adenosine 5′triphosphate (ATP), cytosine 5′triphosphate (CTP), guanosine 5′triphosphate (GTP), uracyle 5′triphosphate (UTP), phosphoenolpyruvate, folic acid, nicotinamide adenine dinucleotide phosphate, pyruvate kinase, adenosine, 3′,5′-cyclic monophosphate (3′,5′cAMP), transfer RNA, amino-acids, amino-acyl tRNA-synthetases, ribosomes, initiation factors, elongation factors and the like. The bacterial cell-free system may further include bacterial or phage RNA polymerase, 70S ribosomes, formyl-methionine synthetase and the like, and other factors necessary to recognize specific signals in the DNA template and in the corresponding mRNA synthesized from said DNA template.

A preferred bacterial cell-free extract is obtained from E. coli cells.

A preferred bacterial cell-free extract is obtained from genetically modified bacteria optimised for cell-free RNA and protein synthesis purposes. As an example, E. coli K12 A19 is a commonly used bacterial strain for cell-free protein synthesis.

The efficiency of the synthesis of proteins in a cell-free synthesis system is affected by nuclease and protease activities, by codon bias, by aberrant initiation and/or termination of translation. In an effort to decrease the influence of these limiting factors and to improve the performance of cell-free synthesis, specific strains can be designed to prepare cell-free extract lacking these non-desirable properties.

It has been shown in the present invention that E. coli BL21Z which lacks Lon and OmpT major protease activities and is widely used for in vivo expression of genes, can also be used advantageously to mediate higher protein yields than those obtained with cell-free extracts from E. coli A19. Thus, one specific embodiment comprises the use of cell-free extracts prepared from E. coli BL21Z.

In bacterial cell-free systems, a major part of the synthesized mRNA are unprotected against hydrolysis and can be subjected to degradation by the RNase E-containing degradosome present in bacterial cell-free extracts. Truncation mutations in the C-terminal or in the internal part of RNase E stabilise transcripts in E. coli cells. Thus, cell-free extracts from E. coli strains which are devoid of RNaseE activity and also protease activity, can be used in cell-free systems for RNA or protein synthesis. Such a strain, E. coli BL21 (DE3) Star, is commercially available from Invitrogen.

The RecBCD nuclease enzymatic complex is a DNA reparation system in E. coli and its activation depends upon the presence of Chi sites (5′GCTGGTGG3′) (SEQ ID NO: 22) on E. coli chromosome. Therefore, a recBCD mutation can be introduced in E. coli host cells in order to decrease the degradation of DNA templates in a cell-free system.

When several codons code for the same amino acid, the frequency of use of each codon by the translational machinery is not identical. The frequency is increased in favor to preferred codons. Actually, the frequency of use of a codon is species-specific and is known as the codon bias. In particular, the E. coli codon bias causes depletion of the internal tRNA pools for AGA/AGG (argU) and AUA (ile Y) codons. By comparing the distribution of synonymous codons in ORFs encoding a protein or RNA of interest and in the E. coli genome, tRNA genes corresponding to identified rare codons can be added to support expression of genes from various organisms. The E. coli BL21 Codon Plus-RIL strain, which contains additional tRNA genes modulating the E. coli codon bias in favor to rare codons for this organism, is commercially available from Stratagene and can be used for the preparation of cell-free extract

Also, improved strains can be used to prevent aggregation of synthesized proteins which can occur in cell-free extracts.

For example, it is well documented that chaperonines can improve protein solubility by preventing misfolding in microbial cytoplasm. In order to decrease a possible precipitation of proteins synthesized in a cell-free system, groES-groEL region can be cloned in a vector downstream an inducible or derepressible promoter and introduced into a E. coli host cell.

Both, protein yield and protein solubility, can further be improved in the presence of homologous or heterologous GroES/GroEL chaperonines in cell-free extracts, prepared from modified E. coli strains, whatever is the selected expression system.

In another embodiment, the cell-free extract is advantageously prepared from cells which overexpress a gene encoding α subunit of RNA polymerase.

Preferred host cells and plasmids used for overexpression of a gene encoding α subunits have been described previously.

Indeed, cell-free extracts prepared from cells overexpressing RNA polymerase α subunit provide improved yield of protein synthesis.

In a preferred embodiment, cell-free extracts are prepared from E. coli strains such as the derivatives of BL21 strain or the E. coli XA 4 strain, overexpressing the rpoA gene.

One advantage of the present embodiment is that the overexpression of α subunit of RNA polymerase is endogeneous and does not need the addition of an exogenous α subunit of RNA polymerase to the reaction mixture. It makes the experimental performance easier and decreases the total cost of in vitro protein synthesis.

It is known in the art that adding purified RNA polymerase may improve the yield of protein synthesis. For example, purified T7 polymerase can be added to the reaction mixture when carrying out cell-free synthesis using a T7 phage promoter. Preferably; adding purified RNA thermostable polymerase, preferably T. thermophilus, in combination with the addition of purified α subunit of RNA polymerase and using bacterial promoter, enables much better yield than with the use of T7 polymerase promoter system.

Thus, in a preferred embodiment, purified thermostable RNA polymerase, preferably from T. thermophilus, is added into a bacterial cell-free extract.

The isolation according to the invention of strong bacterial promoters of bacterial pathogens also provides new approaches for the screening of antibacterial agents which inhibit transcription by binding to strong promoters of said pathogens.

Accordingly, another object of the invention is the use of said isolated nucleic acid having strong bacterial promoter activity for the screening of antibacterial agents which bind to said isolated nucleic acid having strong bacterial promoter activity.

The examples below illustrate some specific embodiments of the invention. Especially, the examples illustrate the identification and isolation of bacterial strong promoters from T. maritima.

### LEGENDS OF THE FIGURES

FIG. 1: A single-strand sequence of putative Thermotoga maritima promoter regions amplified by PCR and the ribosome-binding site used for translation of a reporter gene.

A putative UP-element is shown in italic; putative −35 and −10 sites are underlined; promoter regions putative by algorithm are shown in bold.

A sequence carrying Shine-Dalgarno site GGAGG was placed 12-15 nucleotides downstream the putative −10 site in the corresponding T. maritima promoter. The Shine-Dalgarno site and the ATG initiation codon used for the B. stearothermophilus argC reporter-gene are shown in bold and underlined; additional sequences used to extend the distance between −10 site and Shine-Dalgarno site in tRNAthr1 and TM1016 sequences are shown by lowercase.

FIG. 2: Autoradiogram of ArgC reporter protein synthesis in vitro from DNA templates carrying T. maritima promoter regions.

The B. stearothermophilus argC reporter gene was expressed from putative T. maritima promoter regions or a Ptac promoter in vitro using E. coli S30 extracts. 50 ng of each PCR amplified DNA template was used for in vitro protein synthesis.

Lane 1—Ptac (control); lane 2—PTM0032; lane 3—PTM0373; lane 4—PTM0477; lane 5—PTM1016; lane 6—PTM1067; lane 7—PTM1271; lane 8—PTM1272; lane 9—PTM1429; lane 10—PTM1490; lane 11—PTM1667; lane 12—PTM1780; lane 13—PTARRNAser1; lane 14—PTMtRNAthr1.

FIG. 3: Autoradiogram of ArgC reporter protein synthesis from DNA templates carrying T. maritima promoter regions in the absence and in the presence of α subunit of T. maritima RNA polymerase.

The B. stearothermophilus argC reporter gene was expressed from putative T. maritima promoter regions or a Ptac promoter in the absence (−) or in the presence (+) of 800 nM purified T. maritima RNA polymerase α subunit 50 ng of each PCR amplified DNA template was used for in vitro protein synthesis.

FIG. 4: Autoradiogram of T. maritima ArgG synthesis in the presence and in the absence of α subunit of T. maritima RNA polymerase.

A 1633 bp T. maritima DNA region covering the promoter PargG and the argG gene was amplified by PCR and used for the ArgG protein synthesis in vitro in the absence (lane 1) or in the presence of T. maritima RNA polymerase (X subunit, 400 nM (lane 2) and 800 nM (lane 3).

FIG. 5: Alignment of strong promoter sequences from T. maritima.

The sequence logo for the T. maritima UP element and −5 site was generated with a software at http://www.bio.cam.ac.uk/seqlogo/logo.cqi. An additional N is included into the E. coli UP consensus just before −35 since the residue at this position is not taken into consideration for strong promoter activity in this species.

FIG. 6: Text file presentation of putative strong promoters The data are shown in the Text file with the list of selected strong promoters in the genome with additional information on the operon structure.

FIG. 7: Word form presentation of putative strong promoters In T. maritima genome

FIG. 8: Excell form presentation of putative strong promoters in T. maritima genome

The data are shown with the list of putative strong promoters ordered by their total scores.

### EXAMPLES

A. Material and Methods

A.1 Algorithm for Searching Putative Strong Promoters in Microbial Genomes

A single-strand DNA can be described as a sequence over the four-symbol alphabet {a, c, g, t}, in which a is Adenine, c is Cytosine, g is Guanine and t is Thymine. The DNA length can be measured in nucleotides (nt) for a single-strand molecule or in base pairs (bp) for a double-strand one.

In the present invention, a new algorithm “STRONG_PROMOTERS SEARCH” was developed for searching strong promoters in DNA sequences. Thanks to its flexibility the algorithm can be applied to any microbial genome.

In the present example, a strong bacterial promoter sequence is a DNA region of a size from 44 to 66 bp located upstream the transcription start site of a given gene (coding for protein or tRNA or rRNA sequence), recognized by RNA polymerase holoenzyme containing a major a factor, and which includes three special nucleotide subregions:
1) an UP-element, which is a 17 nt prefix of the strong promoter and has the following consensus pattern “aaaWWtWttttNNNaaa”, where “W” stands for the pair of symbols “a” and “t” and “N” denotes any of four symbols “a”, “c”, and “g”;
2) −35 site, which is located downstream of the UP-element at the distance of 0-5 nt and has the following consensus pattern tcttgacat (underlining marks a commonly used pattern);
3) −10 site, which is located downstream of −35 site at the distance of 14-20 nt and has the following consensus pattern “tataat.

The algorithm uses similarity scores between two sequences, which is the sum of coincidence rates of symbols in the corresponding positions: the equality rate is 1 whereas the nonequality rate is lower than 1 and is determined empirically for each pair of symbols. Therefore, the similarity score of each consensus pattern for any compared sequence varies from 0 to the corresponding length, namely 17 for UP-element, 9 for −35 site and 6 for −10 site.

The algorithm takes as input
1) the name of a genome file in the format GenBank;
2) three parameters of scores: scUP, sc35 and sc10 determining the minimal acceptable value of similarity between UP-element, −35 site and −10 site respectively and the corresponding consensus pattern. Their values 11, 5 and 4 were chosen empirically and are predefined by default, however other values can be input before starting the program.

For each gene in the input genome file, the algorithm runs as follows:
1) first, it extracts an upstream DNA region, namely 300 bp upstream of the corresponding open reading frame or gene-coding for tRNA or rRNA;
2) next, it searches for a strong promoter within this region checking a subregion of the length 70 bp. The algorithm determines the similarity score sUP for the 17 nt prefix with the UP-element consensus pattern (the maximal possible value of sUP is 17) in each identified subregion. If sUP is greater or equal to the given minimal score scUP, then the algorithm checks whether there is an appropriate −35 site downstream of UP-element. In order to obtain the −35 site with the best possible score s35, it uses a special kind of a dynamic programming alignment algorithm, which prohibits any two subsequent insertions or deletions in the −35 consensus pattern and in the chosen DNA subsequence (the maximal possible value of s35 is 9). If s35 is greater or equal to the given minimal score sc35, then the algorithm checks whether there is an appropriate −10 site downstream of −35 site by checking first the distance of 17 nt from the end of −35 site, then by subsequent checking distances of 18, 16, 19, 15, 20 and 14 nt (the maximal possible value of s10 is 6). If s10 is greater or equal to the given minimal score sc10, then the corresponding subregion is included into the list of strong promoters of corresponding genes.
3) For all found strong promoter sequences of each gene, a normalized total score is computed and the best one is output. The normalized total score tot_sc is defined as follows:
   tot_sc=0.30*nsc_up+0.25*nsc_{—}35+0.25*nsc_{—}10+0.2*nsc_dist, where normalized scores nsc_up, nsc_{—}35, nsc_{—}10 are defined by the formulas:
      nsc_{—}up=1−(17−sUP)/20,
      nsc_{—}35=1−(9−s35)/10,
      nsc_{—}10=1−(6−s10)²/10,
   and the values of the normalized distance score nsc_dist are defined in Table 1.

The formulas for nsc_up, nsc_{—}35 and nsc_{—}10 reflect the inexact matching for different subregions. Since −10 site is highly conserved as “tataat” sequence, and then the penalty for each mismatching should be rather high. For example, for 2 mismatches the penalty is (6−4)²/10=0.4 for −10 site, whereas it is (9−6)/10=0.3 for −35 site and (17−15)/20=0.1 for UP-element.

The coefficients 0.30, 0.25 and 0.2 used in the first formula, reflect the relative importance of corresponding subregion for the evaluation of the total score of a strong promoter. They are chosen empirically taking into account the equal significance of −10 and −35 sites, lower significance of the distance between them and higher significance of UP-element. The rate of similarity for each subregion can be modulated by increasing or decreasing the coefficients. However, the set of strong promoters recognized by the developed algorithm doesn't essentially depend on small changes of these coefficients.

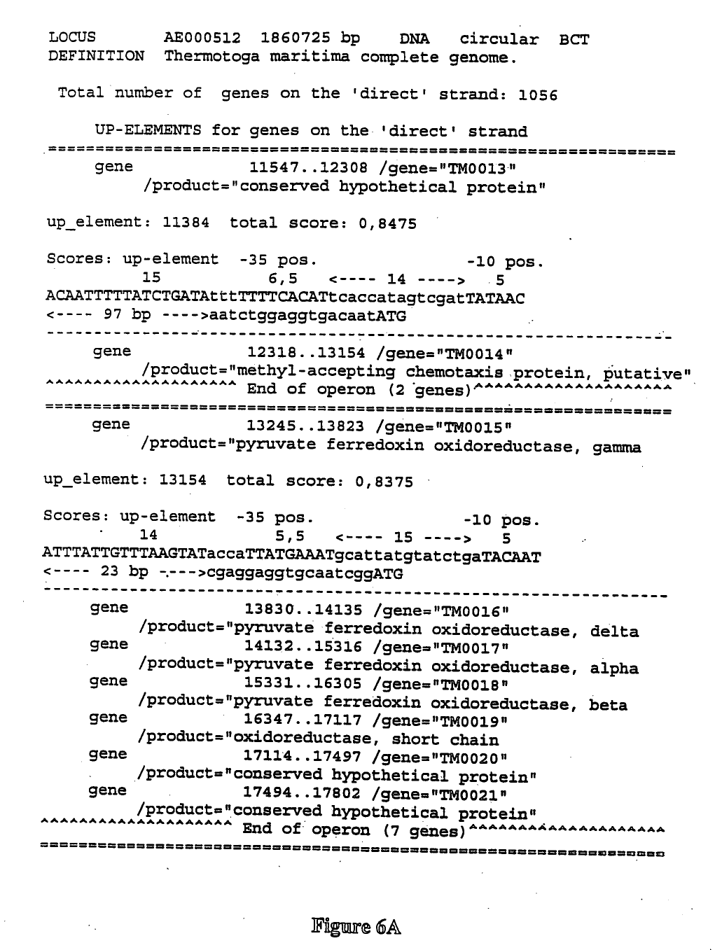

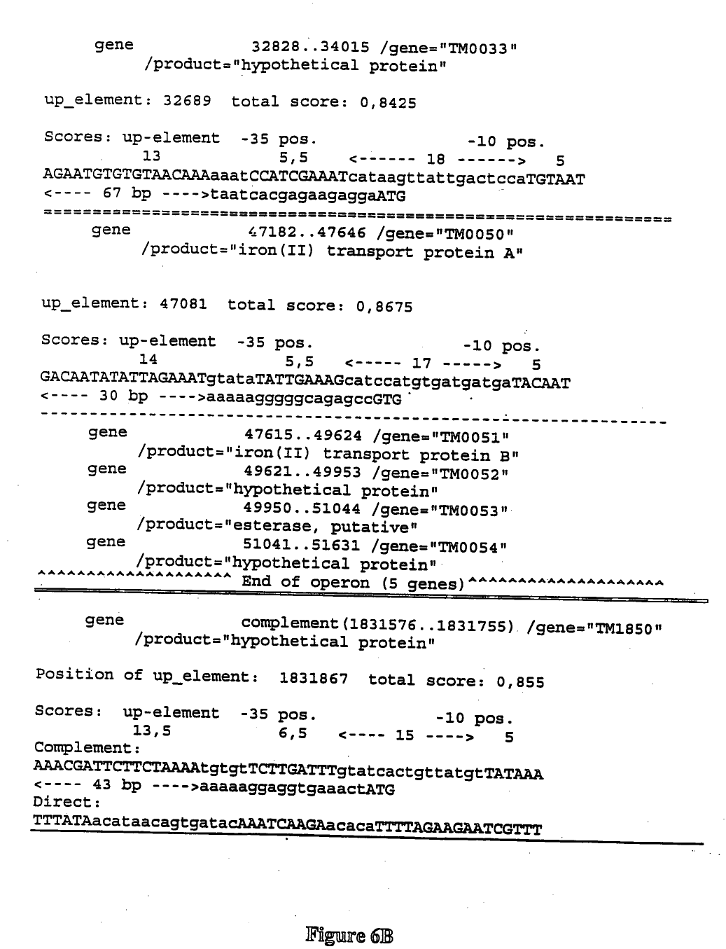

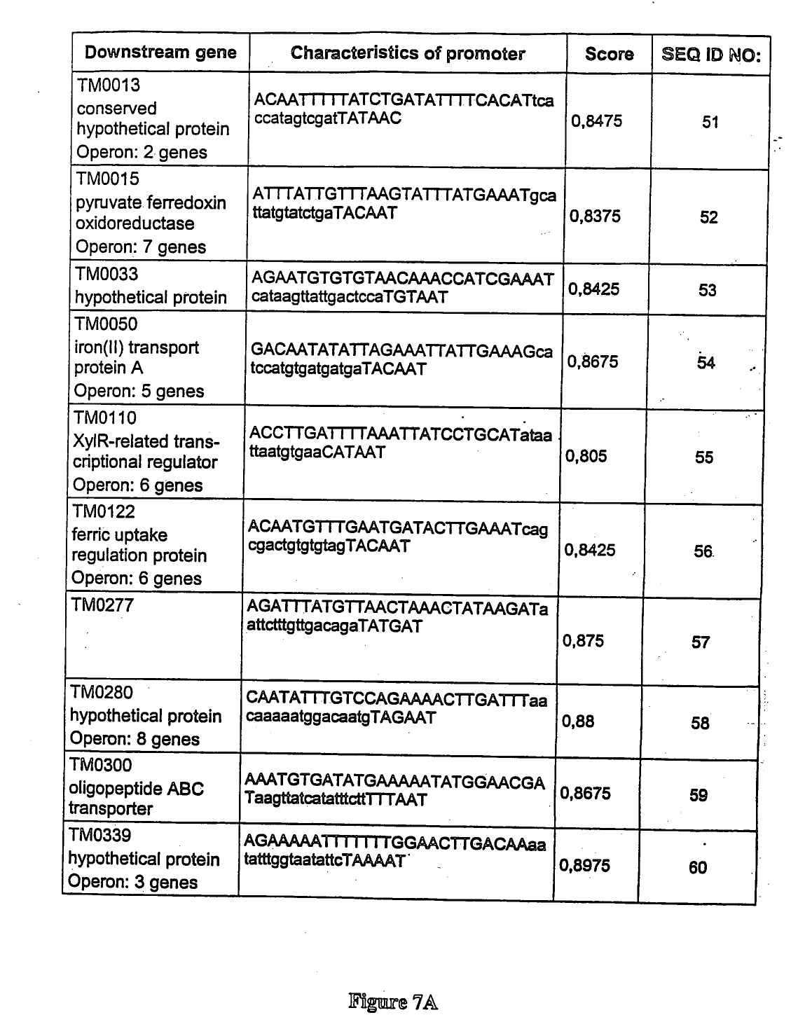

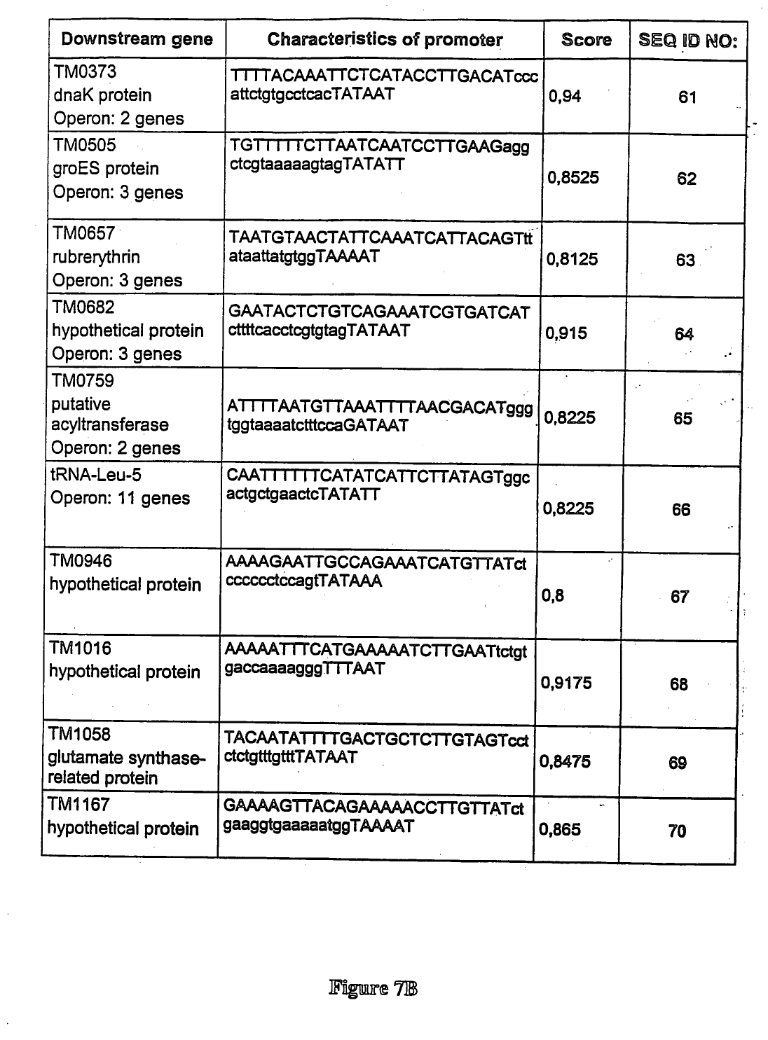

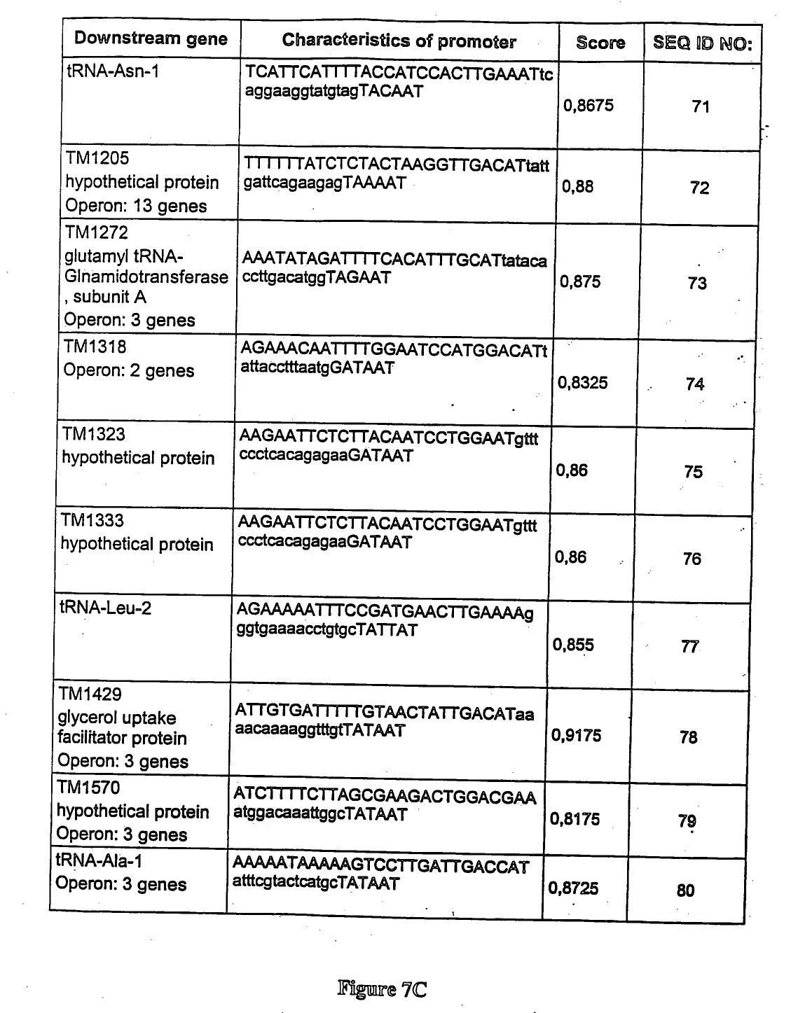

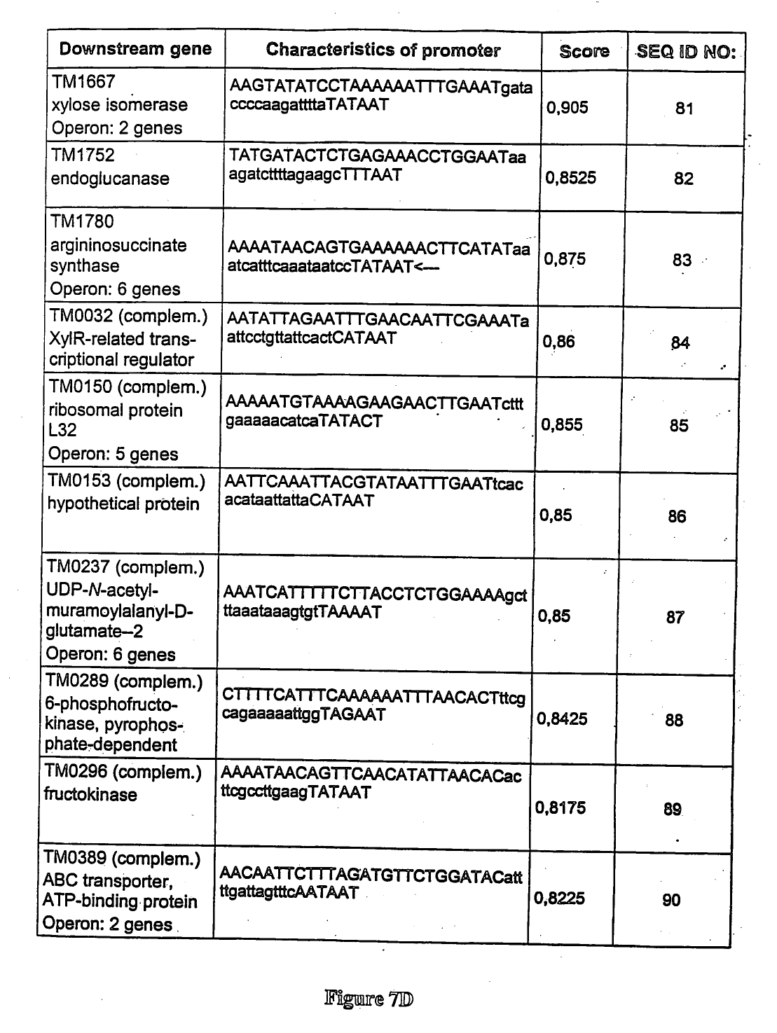

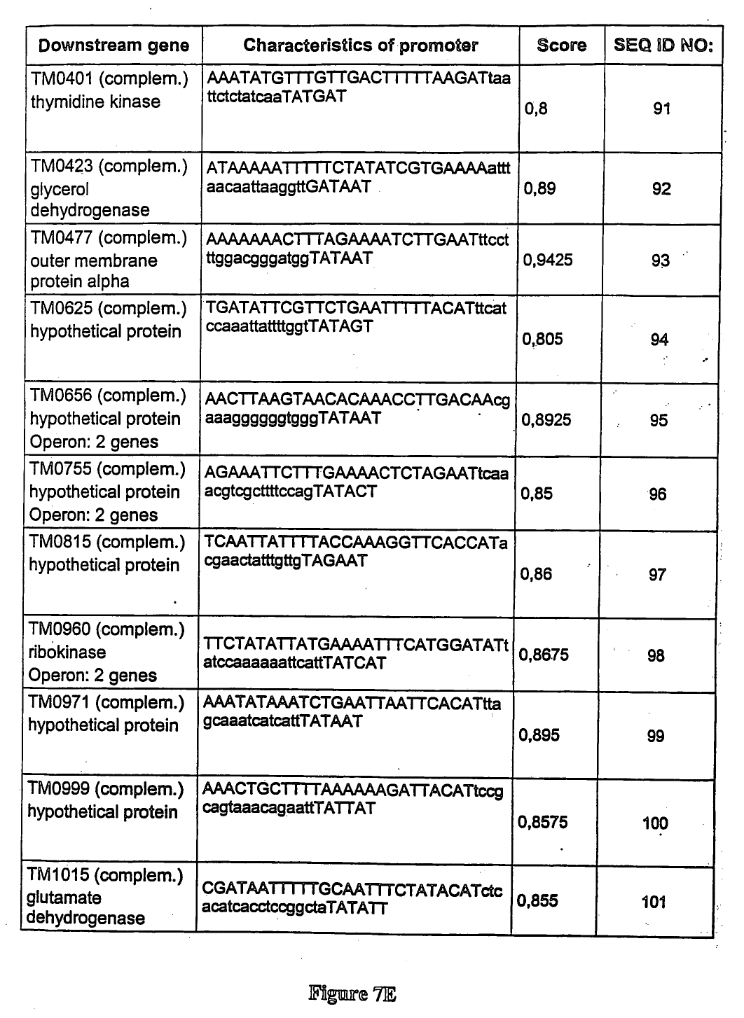

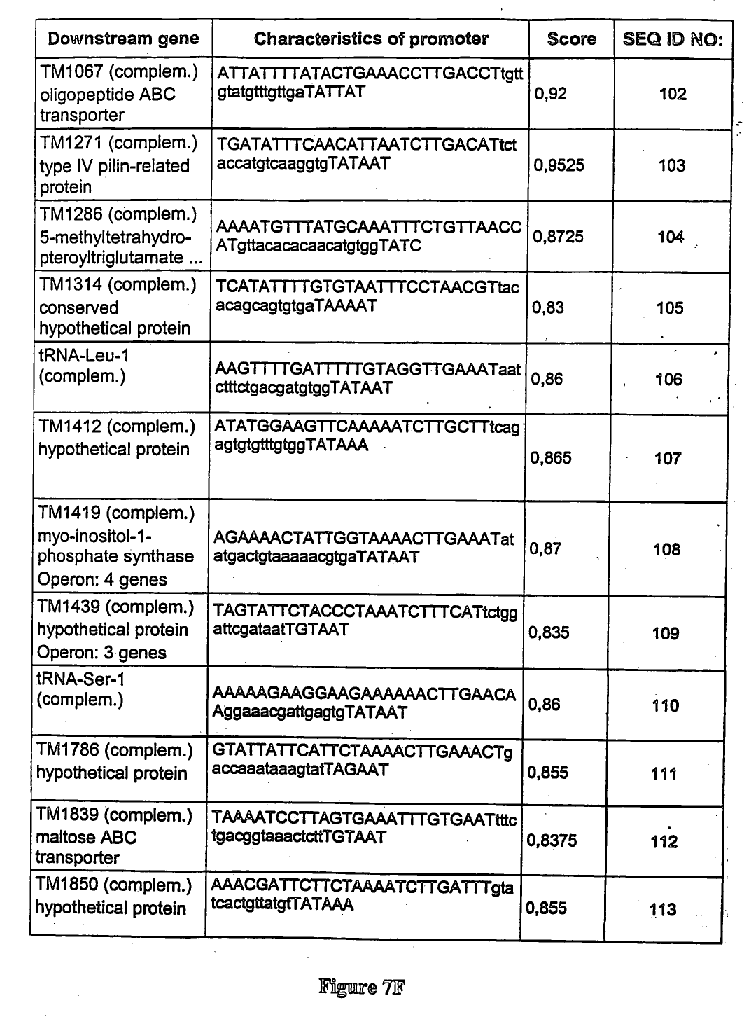

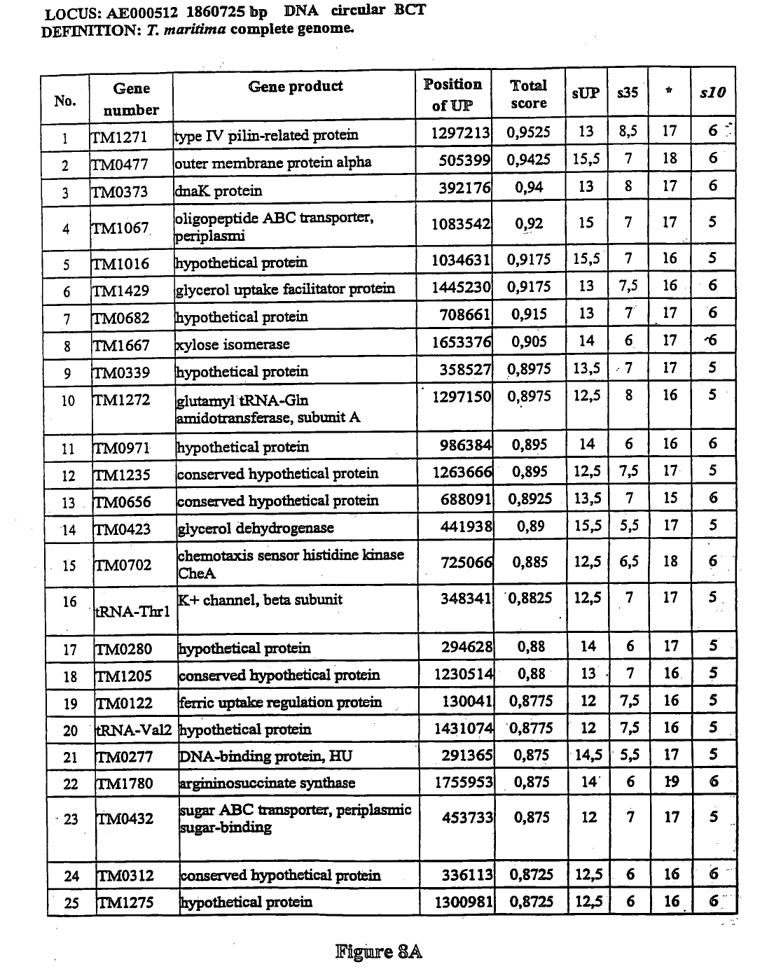

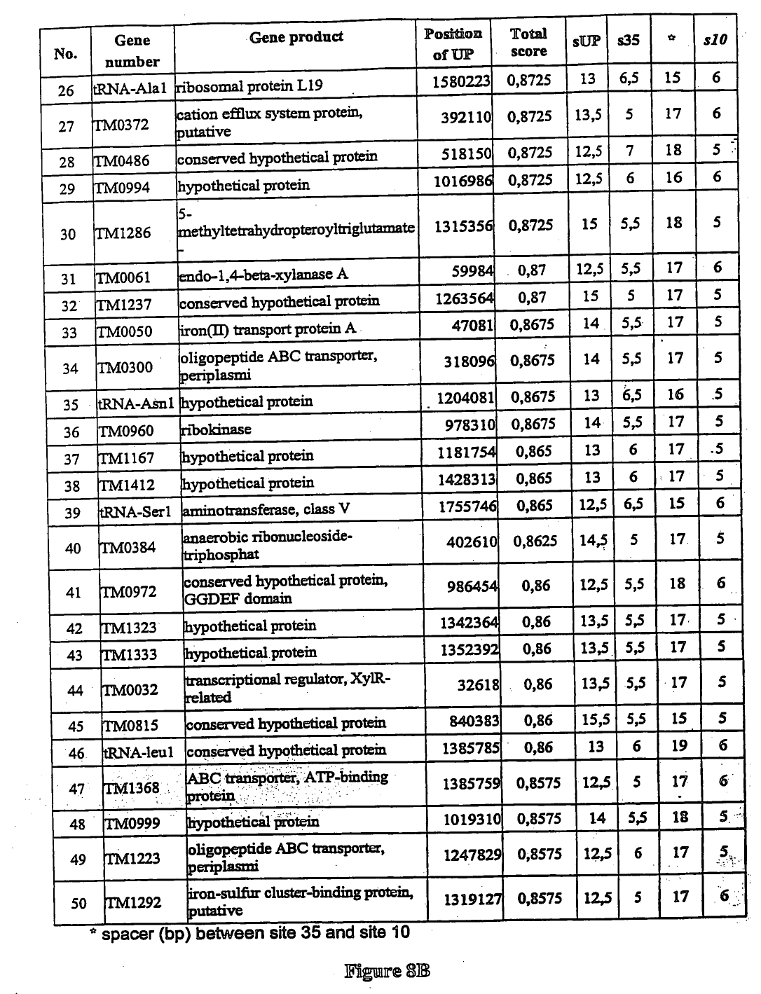

Algorithm “STRONG_PROMOTERS_SEARCH” produces the results in 3 forms:
1) Text-form table with the list of all strong promoters of a genome with additional information on the operons structure (example in FIG. 6);
2) Word-form table with the list of strong promoters (example in FIG. 7);
3) Excel-form table with the list of strong promoters ordered by their total scores (example in FIG. 8).
   A.2 Cloning the rpoA Gene from T. maritima

Chromosomal DNA of the T. maritima MSB8 strain was isolated as described previously (Dimova et al., 2000). A sequence corresponding to the rpoA gene of the RNA polymerase α subunit of T. maritima (Nelson et al., 1999) was amplified on a chromosomal DNA by PCR and two oligonucleotide primers 5′CCATGGCTATAGAATTTGTGATACCAAAAAATTGAGGTG (SEQ ID NO:17) containing the NcoI site and 5′GTCGACTTCCCCCTTCCTGAGCTCAAG (SEQ ID NO:18) containing the Sail site. The amplified DNA fragment was digested by NcoI and SalI and cloned in frame with the C-terminal His-tag sequence of the pET21d+ vector digested by NcoI and XhoI giving rise to pETrpoA. The cloned DNA region with junction sites was verified by automatic DNA sequencing.

A.3 Purification of the Recombinant RNA Polymerase α Subunit of T. maritima

Overexpression of the cloned T. maritima rpoA gene was performed in E. coli BL21 (DE3) (Novagen) by the addition of IPTG (1 mM) to a culture grown up to OD₆₀₀ nm=0.8 and further incubation of cells at 30° C. for 4 hours. The His-tagged RNA polymerase α subunit was next purified from the IPTG-induced culture on a Ni-NTA column by affinity chromatography following a recommended protocol (Qiagen). The purified RNA polymerase α subunit samples were quantified with Lab-on-chip Protein 200 plus assay kit with 2100 Bioanalyzer (Agilent Technologies).

A.4 Construction of DNA Templates for In vitro Synthesis of a Reporter Protein ArgC

The putative promoter regions of T. maritima by the developed algorithm were amplified on chromosomal DNA by PCR using a couple of oligonucleotide primers corresponding to sequences located upstream and downstream of each promoter region. The tac promoter region was also amplified from the plasmid pBTac2 (Bohringer & Mannheim). This chimeric promoter consisting of the native Ptrp and Plac promoters was used as a control strong promoter for comparative analysis of putative T. maritima promoters. Primers used for amplification of promoter regions are described in the following Table 2.

TABLE 2

Oligonucleotide primers used for amplification
of T. maritima promoter regions.

| | | SEQ ID |
|---|---|---|
| Primers | Oligonucleotide séquence | NO: |
| Ptac up | 5′GCGCCGACATCATAACGG | 23 |
| Ptac down | 5′CATATGTTCCCCCTCCTCACAATTCCAC | 24 |
| | ACATTATACC | |
| P0032 up | 5′GCTCCTTGGAAAGAGCATCG | 25 |
| P0032 down | 5′CATATGTTCCCCCTCCTACTCATTTTTT | 26 |
| | ATTATGAG | |
| P0373 up | 5′ATATTCGATTTCCCTCATATTTAGG | 27 |
| P0373 down | 5′CATATGTTCCCCCTCCTCTCATCCATGA | 28 |
| | AAAATTATAG | |
| P0477 up | 5′GAGAGTTGGAAAGAGGAAG | 29 |
| P0477 down | 5′CATATGTTCCCCCTCCTTAAATCCTGTG | 30 |
| | GTGATTAT | |
| P1016 up | 5′CCATATCGTTTACCTATTG | 31 |
| P1016 down | 5′CATATGTTCCCCCTCCCCCGTATGGCTA | 32 |
| | TATATTAAACCCTTTTGG | |
| P1067 up | 5′GGGGTTGTAAGCAAAAGG | 33 |
| P1067 down | 5′CATATGTTCCCCCTCCCTTGAAGTTATC | 34 |
| | AATATAATATC | |
| P1271 up | 5′CGGTTTGTCTTTGAGACGAAT | 35 |
| P1271 down | 5′CATATGTTCCCCCTCCATTTTCACATTT | 36 |
| | TGCATTATAG | |
| P1272 up | 5′CCCGCTCTCTTTCTCATT | 37 |
| P1272 down | 5′CATATGTTCCCCCTCCATTAAAATCTTG | 38 |
| | ACATTCTACC | |
| P1429 up | 5′GAAAGAAGACGTGGAAAG | 39 |
| P1429 down | 5′CATATGTTCCCCCTCCTATGCCTCGATG | 40 |
| | TGAATTATAAC | |
| P1490 up | 5′GCCAGGATAAAGACCATTC | 41 |
| P1490 down | 5′CATATGTTCCCCCTCCACTGTCTTGTCC | 42 |
| | ATTTTATC | |
| P1667 up | 5′CCTCTCTGAGCTCTTCTA | 43 |
| P1667 down | 5′CATATGTTCCCCCTCCTTTTTCTATCAA | 44 |
| | TCAAT | |
| P1780 up | 5′GATATTCATAAACACGAA | 45 |
| P1780 down | 5′CATATGTTCCCCCTCCGTTCTTGATAGC | 46 |
| | ATAATTATAGG | |
| Prna ser1 up | 5′CATCTTTGCACTTTTCG | 47 |
| Prna | 5′CATATGTTCCCCCTCCACACCAGAAAAA | 48 |
| ser1 down | TATTATACAC | |
| Prna thr1 up | 5′TACCAAGGTACGTGGTGA | 49 |
| Prna thr1 | 5′CATATGTTCCCCCTCCCCCGTATGTGCC | 50 |
| down | CGTATGTGTGGTTATTTTAACACACG | |

The sequence used for overlapping between promoter and the reporter argC gene is shown in bold.

The first PCR amplification step was performed with Platinum Pfx DNA polymerase (Invitrogen). Next, the B. stearothermophilus argC gene (Sakanyan et al., 1990; 1993) was used as a reporter to evaluate the strength of isolated promoter regions. In order to increase gene expression an original SD-site of argC was modified from TGAGG to GGAGG. The aryC gene was amplified by PCR using primers argC8-deb (5′-GGAGGGGGAACATATGATGAA) (SEQ ID NO:19) and argCfin-pHav2 (5′-GGACCACCGCGCTACTGCCG) (SEQ ID NO:20) and the obtained DNA fragment was fused downstream of the 13 studied promoters by the overlapping extension” method (Ho et al., 1989). For each construction, the amplified DNAs for a given promoter region of T. maritima and the B. stearothermophilus argC gene region were combined in a subsequent fusion PCR product using two flanking primers by annealing of the overlapped ends to provide a full-length recombinant DNA template. The overlapping region is shown in bold in the used primer sequences (see Table 2). The second PCR reaction was carried by Goldstar Taq DNA polymerase (Eurogentec). The DNA templates obtained by overlapping extension were quantified by Lab-on chip DNA 7500 assay kit with 2100 Bioanalyzer (Agilent Technologies) by injecting 1 μl of a PCR product.

A.5 Preparation of Cell-Free Extracts

A strain E. coli BL21 (DE3) Star RecBCD was used for the preparation of cell-free extracts by the method of Zubay (1973) with modifications as follow:

Cells were grown at 37° C. to OD=0.8, harvested by centrifugation and washed twice thoroughly in ice-cold buffer containing 10 mM Tris-acetate pH 8.2, 14 mM Mg-acetate, 60 mM KCl, 6 mM β-mercaptoethanol. Then, cells were resuspended in a buffer containing 10 mM Tris-acetate pH 8.2, 14 mM Mg acetate, 60 mM KCl, 9 mM dithiotreitol and disrupted by French press (Carver, ICN) at 9 tonnes (≈20.000 psi). The disrupted cells were centrifuged at 30.000 g at 4° C. for 30 min, the pellet was discarded and the supernatant was centrifuged again. The clear lysate was added in a ratio 1:0.3 to the preincubation mixture containing 300 mM Tris-acetate at pH 8.2, 9.2 mM Mg-acetate, 26 mM ATP, 3.2 mM dithiotreitol, 3.2 mM L-amino acids and incubated at 37° C. for 80 min. The mixed extract solution was centrifuged at 6000 g at 4° C. for 10 min, dialysed against a buffer containing 10 mM Tris-acetate pH 8.2, 14 mM Mg-acetate, 60 mM K-acetate, 1 mM dithiotreitol at 4° C. for 45 min with 2 changes of buffer, concentrated 2-4 times by dialysis against the same buffer with 50% PEG-20.000, followed by additional dialysis without PEG for 1 hour. The obtained cell-free extract was distributed in aliquots and stored at −80° C.

A.6 Cell-Free Protein Synthesis by Coupled transcription-Translation Reaction

The coupled transcription-translation reaction was carried out as described by Zubay (−1973) with some modifications. The standard pre-mix contained 50 mM Tris-acetate pH 8.2, 46.2 mM K-acetate, 0.8 mM dithiotreitol, 33.7 mM NH4-acetate, 12.5 mM Mg-acetate, 125 μg/ml tRNA from E. coli (Sigma), 6 mM mixture of CTP, GTP and TTP, 5.5 mM ATP, 8.7 mM CaCl2, 1.9% PEG-8000, 0.32 mM L-amino acids, 5.4 μg/ml folic acid, 5.4 μg/ml FAD, 10.8 μg/ml NADP, 5.4 μg/ml pyridoxin, 5.4 μg/ml para-aminobenzoic acid. Pyruvate was used, as the energy regenerating compound (Kim and Swartz, 1999) by addition of 32 mM pyruvate in 6.7 mM K-phosphate pH 7.5, 3.3 mM thiamine pyrophosphate, 0.3 mM FAD and 6 U/ml pyruvate oxidase (Sigma). Typically, 50 ng of linear PCR-amplified DNA template was added to 25 μl of a pre-mix containing all the amino acids except methionine, 10 μCi of [α³⁵S]-L-methionine (specific activity 1000 Ci/mmol, 37 TBq/mmol, Amersham-Pharmacia Biotech) and E. coli S30 cell-free extracts. The reaction mixture was then incubated at 37-C for 90 min. The purified α subunit of T. maritima RNA polymerase was added to the reaction mixture at different concentrations. The protein samples were treated at 65° C. for 10 min and then quickly centrifuged. The supernatant was precipitated with acetone and used for protein separation on SDS-PAGE, gels were treated with an amplifier solution (Amersham-Pharmacia Biotech), fixed on a 3 MM paper by vacuum drying and the radioactive bands were visualized by autoradiography using BioMax MR film (Kodak). Quantification of cell-free synthesized proteins was performed by counting radioactivity of ³⁵S-labeled ArgC protein with a PhosphorImager 445 SI (Molecular Dynamics).

### B. EXAMPLES

### B.1 Example 1

### Identification of Strong Promoters in T. maritima

As example, the algorithm of “STRONG_PROMOTERS_SEARCH” was used for searching strong promoters in the T. maritima genome. The data are shown in the 3 forms, namely:
1) in the Text file with the list of selected strong promoters in the genome with additional information on the operon structure (FIG. 6A-6B). 33 putative strong promoters identified on a “direct” strand, whereas 30 putative strong promoters were identified on a “complementary” strand.

2) in the Word form with the list of the putative strong promoters (FIG. 7A-7F);
3) in the Excel form with the list of putative strong promoters ordered by their total scores (FIG. 8A-8B).

### B.2 Example 2

### Putative Promoter Sequences of T. maritima Sequences Exhibit a High Activity In vitro

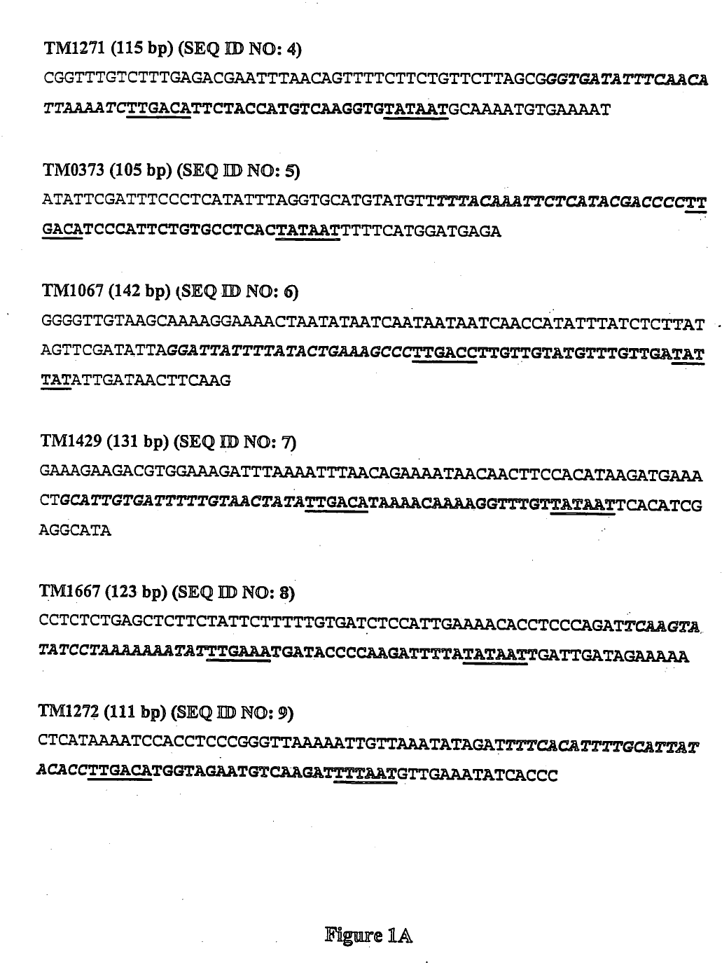

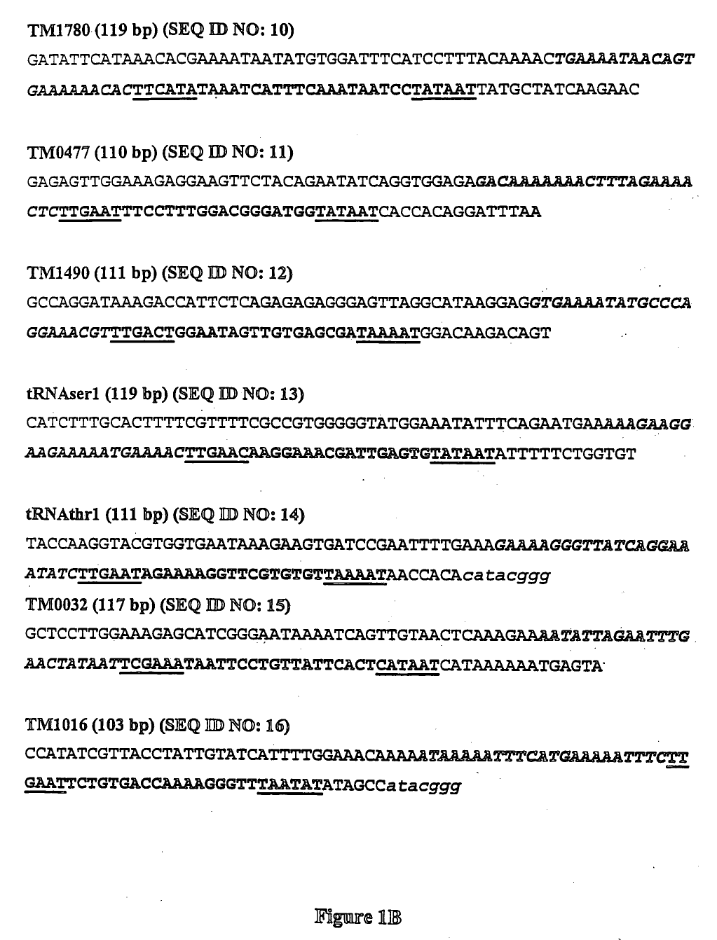

To confirm the presence of functional promoters in the putative T. maritima sequences and to measure the activity of these potential promoters, 13 putative promoter sequences (FIG. 1) were fused to the B. stearothermophilus argC reporter-gene coding for N-acetyl glutamylphosphate reductase. The fused DNA fragments were next used as templates for performing ArgC synthesis in vitro, namely in the coupled E. coli transcription-translation system. Eight sequences were selected from the first 10 selected putative strong promoters shown with a score higher than 0.8975 in FIG. 8. Five others were selected from promoters displaying lower score. The strong Ptac promoter, which has a score of 0.8225 was fused to the reporter gene and used as a reference for comparison with the protein yield provided from T. maritima promoters.

50 ng of such homogen DNA templates, as qualified and quantified by the biochip method, were included into the reaction mixture and protein synthesis was initiated by the addition of S30 extracts.

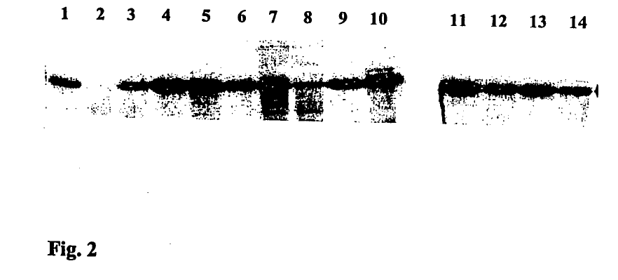

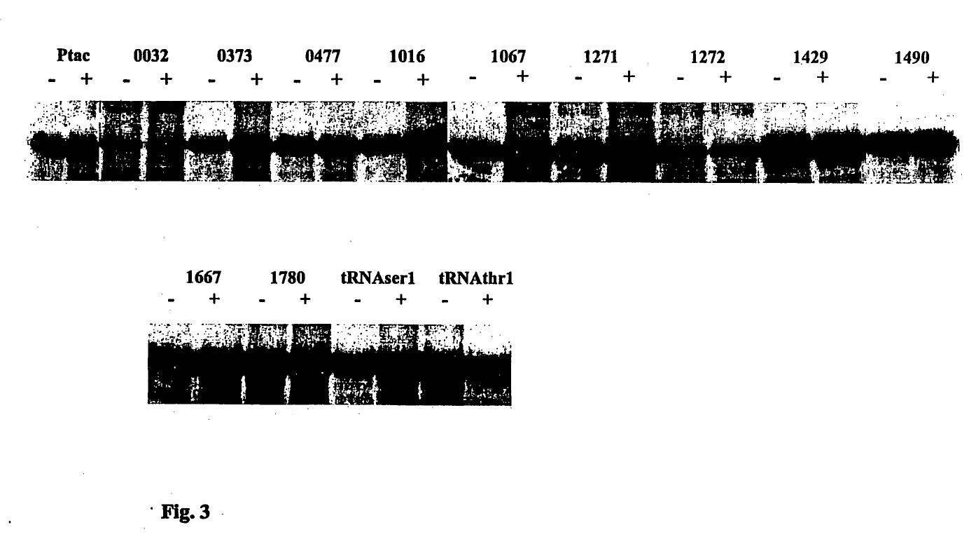

All T. maritima sequences promoted ArgC synthesis as indicative of a presence of functional promoters (FIG. 2). Moreover, all promoter-carrying DNA templates, except for the TM0032 and TM1272 genes, provided higher protein synthesis as compared to the Ptac promoter (the protein yield from the latter was taken as 1 for reference). The 13 selected T. maritima promoters increased the protein yield from 0.5-fold to 2.7-fold (average data from 3 independent experiment) as compared to the Ptac promoter (Table 3).

TABLE 3
T. maritime promoter strength in vitro and the effect of T. maritime
RNA polymerase α subunit on ArgC reporter-protein synthesis

| | | | | Com- | |
|---|---|---|---|---|---|
| Pro- | | | | parative | Effect |
| moter | | Total | | promoter | of α |
| Name | sUP | score | Protein | strength | subunit |
| 1271 | 13 | 0.9525 | Pilin related protein | 2.2 | 1.2 |
| 0477 | 15.5 | 0.9425 | Outer membrane | 2.7 | 2.6 |
| | | | protein α | | |
| 0373 | 13 | 0.9400 | DnaK | 2.1 | 1.5 |
| 1067 | 15 | 0.9200 | ABC transporter | 1.6 | 1.7 |
| | | | periplasmic | | |
| 1016 | 15.5 | 0.9175 | Hypothetical protein | 2.5 | 1.2 |
| 1429 | 13 | 0.9175 | Glycerol uptake | 2.4 | 1.2 |
| | | | facilitator | | |
| 1667 | 14 | 0.9050 | Xylose isomerase | 2.2 | 1.2 |
| 1272 | 12.5/14.5 | 0.8975 | Glutamyl tRNA Gln | 0.9 | 1.7 |
| | | | amidotransferase | | |
| rna thr1 | 12.5 | 0.8825 | tRNA thr1 | 1.7 | 1.2 |
| 1780 | 14 | 0.8750 | ArgG | 2 | 1.2 |
| ma ser1 | 12.5 | 0.8625 | tRNA ser1 | 2.5 | 1.3 |
| 1490 | 12.5 | 0.8450 | Ribosomal protein | 2.1 | 1.2 |
| | | | L14 | | |
| 0032 | 13.5 | 0.8600 | XylR | 0.5 | 2.5 |
| Ptac | 12.5 | 0.8225 | — | 1 | 2.2 |

The high protein yield (more than 2.5-fold) was detected from the promoters identified upstream of TM0477, TM1016 and TMtRNAser1 genes. Eight other putative promoters upstream of TM0373, TM1067, TMtRNAthr1, TM1429, TM1490, TM1667, TM1780 and TM1271 genes increased ArgC synthesis from 1.6-fold to 2.4-fold. It appeared that the identified promoter upstream of TM0032 is subjected to repression by the endogenous E. coli XyIR analogue in S30 extracts.

Thus, E. coli RNA polymerase provided the ArgC reporter-protein in vitro synthesis from the 13 identified T. maritima promoter sequences. Moreover, these results indicate that the identified T. maritima DNA sequences harbour, indeed, strong promoters, which are active in E. coli S30 extracts.

### B.3 Example 3

### T. maritima RNA Polymerase α Subunit Increases the Reporter ArgC Protein Yield In vitro from Putative T. maritima Promoters

Previously It was shown that the addition of E. coli RNA polymerase αsubunit can increase in vitro synthesis of a desired protein expressed from a promoter harbouring a UP-element. Therefore, in this study the effect of the T. maritima RNA polymerase α subunit was also tested on a behaviour of the 13 selected T. maritima promoters in vitro. Indeed, the addition of a purified T. maritima RNA polymerase α subunit, in a range from 800 to 2600 nM, stimulated ArgC synthesis from all promoters (FIG. 3). Quantitative analysis showed that the reporter-gene encoded protein synthesis is increased by 1.2-fold to 2.7-fold as compared in the absence of an exogenous α subunit (Table 3). Protein synthesis was all stimulated from the control strong promoter Ptac in the presence of the T. maritima RNA polymerase α subunit as indicative of the latter's interaction with a heterologous E. coli promoter.

Thus, the data presented indicate that transcription from all tested T. maritima promoters is subjected to the action of homologous RNA polymerase α subunit. Therefore, one should expect that the strength of these promoters is, at least partially, related with the presence of a AT-rich UP element, which is a target for binding RNA polymerase α subunit. The increase of ArgC protein production in vitro by α subunit indicates also that though T. maritima strong promoters are occupied by heterologous E. coli RNA polymerase from S30 extracts, exogenous T. maritima RNA polymerase α subunit can bind to an UP-element of these promoters and provide a higher reporter-gene expression.

### B.4 Example 4

### T. maritima RNA Polymerase α Subunit Increases Protein Yield In vitro from a Native Context of the T. maritima Genome

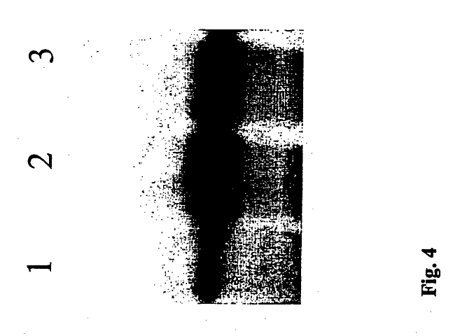

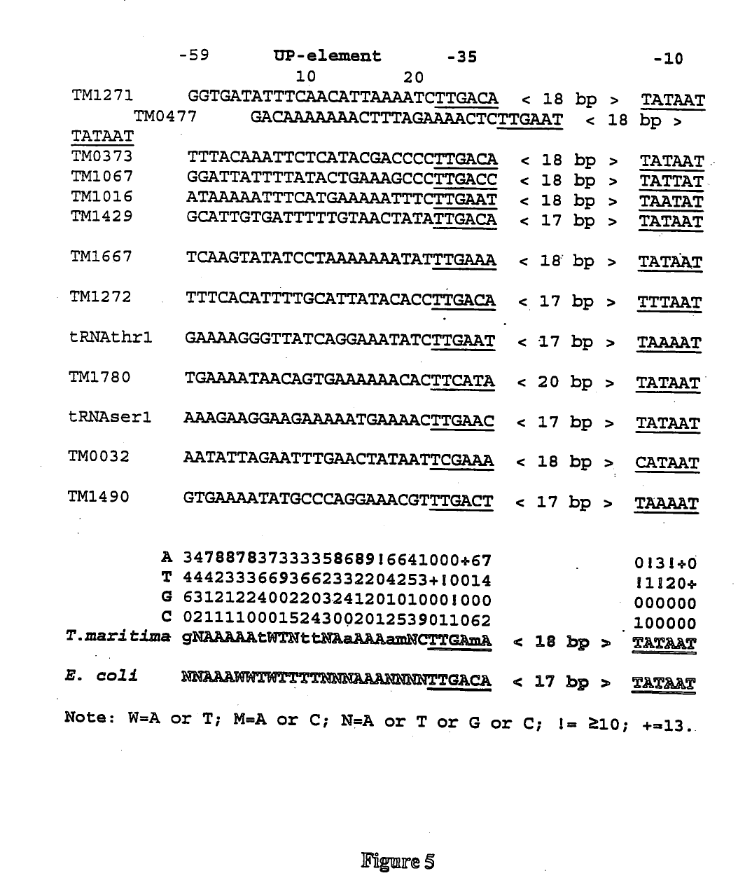

The action of T. maritima RNA polymerase α subunit was also tested on a strong PargG promoter located upstream of TM1780 and governing transcription of a putative argGHJBCD operon of T. maritima by following the ArgG protein synthesis in vitro. The PargG promoter again mediated a high protein production as observed with the reporter-gene argC expression. Moreover, protein synthesis increased nearly 6-fold and 4-fold in the presence respectively, of 500 nM and 1000 nM T. maritima RNA polymerase α subunit (FIG. 4).

### B.5. Example 5

### T. maritima and E. coli UP Elements Possess Differentconsensus Sequences

The 13 strong promoters identified in T. maritima were aligned that permits to characterize corresponding subregions (FIG. 5). The most conserved sequence was found to be −10 site, which is identical to the E. coli consensus (TATAAT) recognized by σ70 factor. A high similarity exists also between −35 site of both bacterial promoters though there is not a preference for the 5^{th} symbol of analysed T. maritima sequences. In strong promoters of this bacterium, −10 and −35 sites are separated by 18 bp rather, than by 17 bp as in E. coli. UP elements of strong promoters from both bacteria also exhibit noticeable similarity as can be judged from two conserved A-tracts (AAA-triplets), which appear to be essential for α subunit contacts and the promoter strength (Gourse et al., 2000). However, UP element of T. maritima strong promoters is richer in Adenine and the distal A-tract appears to be longer in T. maritima than in E. coli. Other possible features are less conserved T-tract in the central part of a full UP element and a preference for Cytosine just before −35 site in strong promoters of T. maritima. It has been supposed that the residue preceding −35 site plays a crucial role in some E. coli strong promoters (Estrem et al., 1999). As in E. coli the T. maritima UP element's AAA-triplets are separated by 11 bp supposing that the same surface of two α subunits determines DNA contacts. However, the presence of longer A-tracts in T. maritima allows to assume more dynamism in the capacity of its RNA polymerase to recognise corresponding UP element subsites upstream of −5 consensus.

Thus, the detected features between strong promoter sequences of the two bacteria allow assuming that RNA polymerase-promoter interactions can be somehow different in distant bacteria.

### B.6 Example 6

### Identification of Strong Promoters in Other Sequenced Bacterial Genomes

Next, the algorithm “STRONG_PROMOTERS_SEARCH” was used to identify strong promoters in 46 available bacterial genomes in GenBank (Table 4).

TABLE 4
Number of putative strong promoters in bacterial genomes.

| | | | Number | | |
|---|---|---|---|---|---|
| N^{o} | Genome | Length, bp | of genes | * | ** |
| 1 | Deinococcus radiodurans | 2648638 | 2681 | 5 | 1 |
| | R1 (AE000513) | | | | |
| 2 | Pseudomonas aeruginosa | 6264403 | 5570 | 15 | 2 |
| | PA01 (AE004091) | | | | |
| 3 | Mycobacterium | 4411529 | 3922 | 7 | 0 |
| | tuberculosis (AL123456) | | | | |
| 4 | Caulobacter crescentus | 4016947 | 3787 | 2 | 0 |
| | (AE005673) | | | | |
| 5 | Ralstonia solanacearum | 3716413 | 3477 | 7 | 0 |
| | GMI1000 chromosome | | | | |
| | (AL646052) | | | | |
| 6 | Xanthomonas compestris | 5076188 | 4197 | 2 | 0 |
| | pv. campestris str. ATCC | | | | |
| | 33913 (AE008922) | | | | |
| 7 | Xanthomonas axonopodis | 5175554 | 4344 | 2 | 0 |
| | pv. citri str. 306 | | | | |
| | (AE008923) | | | | |
| 8 | Mesorhizobium loti | 7036074 | 6693 | 9 | 0 |
| | NC_002670) | | | | |
| 9 | Sinorhizobium meliloti | 3654135 | 3375 | 8 | 0 |
| | 1021 (AL591688) | | | | |
| 10 | Mycobacterium leprae | 3268203 | 2770 | 8 | 1 |
| | strain TN (AL450380) | | | | |
| 11 | Agrobacterium | 2074782 | 1825 | 12 | 3 |
| | tumefaciens strain C58 | | | | |
| | linear chromosome | | | | |
| | (AE007870) | | | | |
| 12 | Brucella melitensis strain | 2117144 | 2059 | 21 | 4 |
| | 16M chromosome I | | | | |
| | (AE008917) | | | | |
| 13 | Agrobacterium | 2841581 | 2701 | 20 | 1 |
| | tumefaciens strain C58 | | | | |
| | circular chromosome | | | | |
| | (AE007869) | | | | |
| 14 | Treponema pallidum | 1138011 | 1083 | 4 | 3 |
| | (AE000520) | | | | |
| 15 | Chlorobium tepidum TLS | 2154946 | 2329 | 35 | 13 |
| | (AE006470) | | | | |
| 16 | Salmonella typhimurium | 4857432 | 4608 | 163 | 61 |
| | LT2 (AE006468) | | | | |
| 17 | Neisseria meningitidis | 2272351 | 2226 | 112 | 45 |
| | serogroup B strain MC58 | | | | |
| | (AE002098) | | | | |
| 18 | Escherichia coli 0157:H7 | 5528445 | 5478 | 263 | 79 |
| | (AE005174) | | | | |
| 19 | Xylella fastidiosa plasmid | 51158 | 64 | 4 | 0 |
| | pXF51 (AE003851) | | | | |
| 20 | Vibrio cholerae | 2961149 | 2887 | 93 | 37 |
| | chromosome I | | | | |
| | (AE003852) | | | | |
| 21 | Yersinia pestis strain | 4653728 | 4042 | 274 | 61 |
| | CO92 (AL590842) | | | | |
| 22 | Methanobacterium | 1751377 | 1900 | 81 | 24 |
| | thermoautotrophicum | | | | |
| | delta H (AE000666) | | | | |
| 23 | Synechocystis PCC6803 | 3573470 | 1074 | 31 | 6 |
| | (AB001339) | | | | |
| 24 | Thermotoga maritima | 1860725 | 1926 | 63 | 10 |
| | (AE000512) | | | | |
| 25 | Aquifex aeolicus | 1551335 | 1503 | 71 | 37 |
| | (AE000657) | | | | |
| 26 | Bacillus halodurans C-125 | 4202353 | 4125 | 359 | 87 |
| | (BA000004) | | | | |
| 27 | Bacillus subtilis | 4214814 | 4182 | 430 | 111 |
| | (AL009126) | | | | |
| 28 | Chlamydia muridarum | 1069411 | 954 | 86 | 31 |
| | (AE002160) | | | | |
| 29 | Mycoplasma pneumoniae | 816394 | 705 | 37 | 14 |
| | M129 (U00089) | | | | |
| 30 | Streptococcus | 2160837 | 2306 | 365 | 156 |
| | pneumoniae (AE005672) | | | | |
| 31 | Helicobacter pylori, strain | 1643831 | 1495 | 182 | 54 |
| | J99 (AE001439) | | | | |
| 32 | Streptococcus pyogenes | 1852441 | 1731 | 292 | 115 |
| | strain SF370 serotype M1 | | | | |
| | (AE004092) | | | | |
| 33 | Haemophilus influenzae | 1830138 | 1775 | 277 | 94 |
| | Rd (L42023) | | | | |
| 34 | Pasteurella multocida | 2257487 | 1996 | 228 | 64 |
| | PM70 (AE004439) | | | | |
| 35 | Listeria innocua | 3011208 | 3529 | 426 | 229 |
| | Clip11262 (AL592022) | | | | |
| 36 | Chlamydophila | 1226565 | 1097 | 162 | 51 |
| | pneumoniae J138 | | | | |
| | (BA000008) | | | | |
| 37 | Thermoanaerobacter | 2689445 | 2632 | 467 | 248 |
| | tengcongensis strain | | | | |
| | MB4T (AE008691) | | | | |
| 38 | Clostridium | 3940880 | 3738 | 1685 | 916 |
| | acetobutylicum ATCC824 | | | | |
| | (AE001473) | | | | |
| 39 | Mycoplasma genitalium | 580074 | 519 | 83 | 63 |
| | G37 (L43937) | | | | |
| 40 | Staphylococcus aureus | 2814816 | 2638 | 930 | 418 |
| | strain N315 (BA000018) | | | | |
| 41 | Rickettsia prowazekii | 1111523 | 885 | 443 | 252 |
| | strain Madrid E | | | | |
| | (AJ235269) | | | | |
| 42 | Campylobacter jejuni | 1641481 | 1684 | 540 | 353 |
| | (AL111168) | | | | |
| 43 | Lyme disease spirochete, | 910724 | 875 | 350 | 292 |
| | Borrelia | | | | |
| | burgdorferi.(AE000783) | | | | |
| 44 | Clostridium perfringens | 3031430 | 2779 | 1499 | 772 |
| | 13 DNA (BA000016) | | | | |
| 45 | Ureaplasma urealyticum | 751719 | 645 | 328 | 236 |
| | (AF222894) | | | | |
| 46 | Buchnera aphidicola str. | 641454 | 584 | 339 | 225 |
| | Sg (Schizaphis graminum) | | | | |
| | (AE013218) | | | | |

* Number of putative strong promoter sequences in “upstream” regions
** Number of putative strong promoters in “downstream” regions

The table 4 shows the number of strong promoters putative for each genome. For comparison it includes the number of false strong promoter-like” regions detected downstream of real promoter regions, namely a search for a 300 bp region after the transcription start site of all genes by the algorithm. The results clearly indicate that the number of strong promoter-like sequences differ dramatically in 300 bp portion located upstream and downstream of the corresponding regions, thereby confirming the validity of at least majority of the identified sequences on a genome scale.

### B.7 Example 7

### Number of Strong Promoters Reflects an A+T Composition of Bacterial Genomes

Since 24 of 29 symbols in all three patterns are a's and t's one can suppose that the percentage of genes with strong promoters depends on the percentage of symbols a and t in a given genome. The computational experiments confirm partially this assumption (Table 5).

TABLE 5
Relation between number of putative strong promoters
and A + T composition of bacterial genomes

| | | | strong | random |
|---|---|---|---|---|
| N° | Bacterial genome | at % | promoters % | s.p. % |
| 1 | Deinococcus radiodurans R1 | 32.99 | 0.19 | 0 |
| | (AE000513) | | | |
| 2 | Pseudomonas aeruginosa | 33.44 | 0.27 | 0 |
| | PA01 (AE004091) | | | |
| 3 | Mycobacterium tuberculosis | 34.39 | 0.18 | 0 |
| | (AL123456) | | | |
| 4 | Caulobacter crescentus | 34.40 | 0.05 | 0 |
| | (AE005673) | | | |
| 5 | Ralstonia solanacearum | 34.51 | 0.20 | 0 |
| | GMI1000 chromosome | | | |
| | (AL646052) | | | |
| 6 | Xanthomonas campestris pv. | 35.64 | 0.05 | 0 |
| | campestris str. ATCC 33913 | | | |
| | (AE008922) | | | |
| 7 | Xanthomonas axonopodis pv. | 36.02 | 0.05 | 0 |
| | citri str. 306 (AE008923) | | | |
| 8 | Mesorhizobium loti | 39.09 | 0.13 | 0 |
| | NC_002670) | | | |
| 9 | Sinorhizobium meliloti 1021 | 39.66 | 0.24 | 0 |
| | (AL591688) | | | |
| 10 | Mycobacterium leprae strain | 42.20 | 0.29 | 0 |
| | TN (AL450380) | | | |
| 11 | Agrobacterium tumefaciens | 42.68 | 0.66 | 0 |
| | strain C58 linear chromosome | | | |
| | (AE007870) | | | |
| 12 | Brucella melitensis strain 16M | 42.84 | 1.02 | 0 |
| | chromosome I (AE008917) | | | |
| 13 | Agrobacterium tumefaciens | 43.20 | 0.74 | 0 |
| | strain C58 circular | | | |
| | chromosome (AE007869) | | | |
| 14 | Treponema pallidum | 47.01 | 0.37 | 0 |
| | (AE000520) | | | |
| 15 | Chlorobium tepidum TLS | 47.50 | 1.50 | 0.303 |
| | (AE006470) | | | |
| 16 | Salmonella typhimurium LT2 | 47.78 | 3.54 | 0.306 |
| | (AE006468) | | | |
| 17 | Neisseria meningitidis | 48.47 | 5.03 | 0.33 |
| | serogroup B strain MC58 | | | |
| | (AE002098) | | | |
| 18 | Escherichia coli O157:H7 | 49.50 | 4.80 | 0.48 |
| | (AE005174) | | | |
| 19 | Xylella fastidiosa plasmid | 51.43 | 6.25 | 0.84 |
| | pXF51 (AE003851) | | | |
| 20 | Vibrio cholerae chromosome I | 52.30 | 3.22 | 1 |
| | (AE003852) | | | |
| 21 | Yersinia pestis strain CO92 | 52.36 | 6.78 | 1.08 |
| | (AL590842) | | | |
| 22 | Methanobacterium | 53.11 | 4.26 | 1.28 |
| | thermoautotrophicum delta H | | | |
| | (AE000666) | | | |
| 23 | Synechocystis PCC6803 | 53.71 | 2.89 | 1.7 |
| | (AB001339) | | | |
| 24 | Thermotoga maritima | 53.75 | 3.27 | 1.75 |
| | (AE000512) | | | |
| 25 | Aquifex aeolicus (AE000657) | 57.73 | 4.72 | 4.05 |
| 26 | Bacillus halodurans C-125 | 58.65 | 8.70 | 4.75 |
| | (BA000004) | | | |
| 27 | Bacillus subtilis (AL009126) | 59.30 | 10.28 | 5.7 |
| 28 | Chlamydia muridarum | 59.69 | 9.01 | 6.6 |
| | (AE002160) | | | |
| 29 | Mycoplasma pneumoniae | 59.99 | 5.25 | 7.35 |
| | M129 (U00089) | | | |
| 30 | Streptococcus pneumoniae | 60.30 | 15.83 | 7.7 |
| | (AE005672) | | | |
| 31 | Helicobacter pylori, strain J99 | 60.81 | 12.17 | 8.35 |
| | (AE001439) | | | |
| 32 | Streptococcus pyogenes | 61.49 | 16.87 | 9.5 |
| | strain SF370 serotype M1 | | | |
| | (AE004092) | | | |
| 33 | Haemophilus influenzae Rd | 61.85 | 15.61 | 9.9 |
| | (L42023) | | | |
| 34 | Pasteurella multocida PM70 | 62.31 | 11.42 | 10.9 |
| | (AE004439) | | | |
| 35 | Listeria innocua Clip11262 | 62.56 | 12.07 | 11.5 |
| | (AL592022) | | | |
| 36 | Chlamydophila pneumoniae | 62.80 | 14.77 | 12.5 |
| | J138 (BA000008) | | | |
| 37 | Thermoanaerobacter | 64.11 | 17.74 | 14.8 |
| | tengcongensis strain MB4T | | | |
| | (AE008691) | | | |
| 38 | Clostridium acetobutylicum | 69.07 | 45.08 | 32.9 |
| | ATCC824 (AE001473) | | | |
| 39 | Mycoplasma genitalium G37 | 69.50 | 15.99 | 35 |
| | (L43967) | | | |
| 40 | Staphylococcus aureus strain | 69.71 | 35.25 | 35.5 |
| | N315 (BA000018) | | | |
| 41 | Rickettsia prowazekii strain | 71.00 | 50.06 | 40.2 |
| | Madrid E (AJ235269) | | | |
| 42 | Campylobacter jejuni | 71.36 | 32.07 | 41.8 |
| | (AL111168) | | | |
| 43 | Lyme disease spirochete, | 71.40 | 40.00 | 42 |
| | Borrelia | | | |
| | burgdorferi.(AE000783) | | | |
| 44 | Clostridium perfringens 13 | 71.43 | 53.94 | 42.1 |
| | DNA (BA000016) | | | |
| 45 | Ureaplasma urealyticum | 76.05 | 50.85 | 65.35 |
| | (AF222894) | | | |
| 46 | Buchnera aphidicola str. Sg | 78.36 | 58.05 | 74.5 |
| | (Schizaphis graminum) | | | |
| | (AE013218) | | | |

The third column “at %” shows the percentage of symbols a and t into genomes, the next column “strong promoters %” shows the percentage of genes with strong promoters among all genes of genomes. The following score parameters where used: scup=13.0, sc35=5.5, sc10=5.0. The last column shows the percentage of genes with strong promoters among random upstream regions which where generated with the same percentage of a's and f's as in the corresponding “real” genomes.

This table shows that genomes with rather small percentage a's and t's (less than 50%) have much more genes transcribed from strong promoters as compared from “random genomes” with a similar percentage a's and f's. When percentage a's and f's grows from 50% to 65% the difference between the percentage of strong promoters into real and random genomes decreases but still is meaningful enough. However, this difference disappears when the percentage a's and f's exceeds 65%. There are some exceptions. For example, three tested mycoplasmial genomes (data are shown for a single representative) have relatively low percentage of genes transcribed from strong promoter.

Thus, the developed algorithm permits to identify strong putative promoters in bacterial genomes. The algorithm is based on the identification of promoters containing an UP-element and conservative −10 and −35 sites separated by 17 bp. The putative highly expressed bacterial genes can be clustered into several groups, which include essential for cellular growth genes for translation, protein transport and protein folding as well as “non-essential” or non-yet identified ones. It appears that functions of “non-essential” genes are related with providing large quantities of encoded proteins required to adapt to various extra-cellular environmental conditions.

The strength of putative promoters has been proven experimentally for 13 putative promoter sequences of a hyperthermophilic bacterium T. maritima using a reporter-gene expression from a linear DNA template in a coupled transcription-translation system. Though such an evaluation may diminish a real promoter strength because of gene expression by a heterologous RNA polymerase holoenzyme, but the proposed approach avoids time-consuming steps for DNA cloning in cells. The method can be especially useful for simultaneous and rapid characterization of numerous putative promoters in bacterial genomes, including pathogens. All T. maritima promoters wee found to mediate high protein synthesis in vitro. Moreover, the addition of the purified α subunit of T. maritima or E. coli RNA polymerase increases the protein yield from all tested promoters, thereby proving the essential role of RNA polymerase α subunit/UP element interactions for determining the promoter strength. Indeed, this subunit is able to bind the promoter sequences as shown by the protein array method for several cases.

The data presented show that the behaviour of some strong promoters depends on interactions with heterologous transcription regulatory proteins in E. coli S30 extracts that appears to prohibit binding α subunit of T. maritima RNA polymerase to DNA targets and, thereby decrease protein expression.

The identified strong promoters from various bacterial sources can be used both for the construction of new expression vectors and protein overproduction in cellular and cell-free systems.

Furthermore, the Identified strong promoters in pathogenic bacteria, for example in Mycobacterium tuberculosis, Mycobacterium leprae, Pseudomonas aeruginosa, Brucella melitensis, Neisseria meningitis, Salmonella typhimurium, Escherichia coli, Vibrio cholerae, Yersinia pestis, Streptococcus pneumoniae, Streptococcus pyogenes, Haemophilus influenzae and Helicobacter pylori are also attractive as potential targets for development of new antibacterial therapy approaches.

### REFERENCES

Aiyar, S. E., Gourse, R. L. & Ross, W. (1998). Upstream A-tracts increase bacterial promoter activity through interactions with the RNA polymerase alpha subunit. Proc. Natl; Acad. Sci. USA 95, 14652-14657.
Aiyar, S. E., Gaal, T. & Gourse, R. L. (2002). rRNA promoter activity in the fast-growing bacterium Vibrio natrigens. J. Bacter. 184, 1349-1358.
Altschul S., Gish W., Miller E., Myers E., and Lipman J. (1990). Basic local alignment search tool. J. Mol. Biol. 215, 403-410.
Chen, G., Dubrawski, I., Mendez, P., Georgiou, G. & Iverson, B. L. (1999). In vitro scanning saturation mutagenesis of all the specificity determining residues in an antibody binding site. Protein Eng. 12, 349-356.
Dimova D., Weigel P., Takahashi M., Marc F., Van Duyne G. D. & Sakanyan, V. (2000). Thermostability, oligomerisation and DNA-binding properties of the regulatory protein ArgR from the hyperthermophilic bacterium Thermotoga neapolitana. Mol. Gen. Genet. 263,119-130.
Estrem, S. T., Gaal, T., Ross, W. & Gourse, R. L. (1998). Identification of an UP element consensus sequence for bacterial promoters. Proc. Natl. Acad. Sci. USA 95, 9761-9766.
Estrem, S. T., Ross, W., Gaal, T., Chen, Z W. S. I, Niu, W., Ebright, R H. & Gourse, R. L. (1999). Bacterial promoter architecture: subsite structure of UP elements and interactions with the C-terminal domain of the RNA polymerase α subunit. Genes & Dev. 13, 2134-2147.
Fredrick, K., Caramori, T., Chen, Y. F., Galizzi, A& Helmann, J. D. (1995). Promoter architecture in the flagellar regulon of Bacillus subtillis: high-level expression of flagellin by the sigma D RNA polymerase requires an upstream promoter element. Proc. Natl. Acad. Sci. USA 92, 2582-2586.
Gourse, R. L., Ross, W. & Gaal, T. (2000). Ups and downs in bacterial transcription initiation: the role of the alpha subunit of RNA polymerase in promoter recognition. Mol. Microbiol 37, 687-695.
Graves, M. C. & Rabinowitz, J. C. (1986). In vivo and in vitro transcription of the Clostridium pasterianum ferredoxin gene. Evidence for “extended” promoter elements in gram-positive organisms. J. Biol. Chem. 261, 11409-11415.
Ho, N. S., Hunt, D. H., Horton, M. R., Pullen K. J. & Pease R., L. (1989). Site directed mutagenesis by overlap extension using the polymerase chain reaction. Gene 77, 51-59.
Kigawa, T., Yabuki, T., Yoshida, Y., Tsutsui, M., Ito, Y., Shibata, T. & Yokoyama, S. (1999). Cell-free production and stable-isotope labeling of milligram quantities of proteins. FEBS Letters 442, 15-19.
Kim, D.-M. & Swartz, J. R. (1999). Prolonging cell-free protein synthesis with a novel ATP regeneration system. Biotech. & Bioengin. 66, 180-188.
Kimura, M. & Ishihama, A. (1996). Subunit assembly in vivo of Escherichia coli RNA polymerase: role of the amino-terminal assembly domain of alpha subunit Genes Cells 1, 517-28.
Lesley, S. S., Borw, M. A. & Burgess, R. R. (1991). Use of in vitro protein synthesis from polymerase chain reaction-generated templates to study interaction of Escherichia coli transcription factors with core RNA polymerase and for epitope mapping of monoclonal antibodies. J. Biol. Chem. 266, 2632-2638.
Mattheakis, L. C., Dias, J. M. & Dower, W. J. (1996). Cell-free synthesis of peptide libraries displaied on polysomes. Meth. Enzymol. 267, 195-207.
Nelson, K. E. et al. (1999). Evidence for lateral gene transfer between Archaea and Bacteria from genome sequence of Thermotoga maritima. Nature 399, 323-329.
Pelham, H. R. & Jackson, R. J. (1976). An efficient mRNA-dependent translation system from reticulocyte lysates. Eur. J. Biochem. 67, 247-256.
Roberts, B. E. & Paterson, B. M. (1973). Efficient translation of tobacco mosaic virus RNA and rabbit globin 9S RNA in a cell-free system from commercial wheat germ. Proc. Natl. Acad. Sci. USA 70, 2330-2334.
Ross, W., Gosink, K. K., Salomon, J., Igarashi, K., Zou, C., Ishihama, A, Severinov, K. & Gourse, R. L. (1993). A third recognition element in bacterial promoters: DNA binding by the α subunit of RNA polymerase. Science 262, 1407-1413.
Ross, W., Ernst, A. & Gourse, R. L. (2001). Fine structure of E. coli RNA polymerase-promoter interactions: α subunit binding to the UP element minor groove. Genes & Dev. 15, 491-506.
Sambrook et al. (2001). Molecular Cloning: A laboratory Manual, 3^{rd} Ed., Cold Spring Harbor, laboratory press, Cold Spring Harbor, N.Y.
Sakanyan, V. A., Hovsepyan, A. S., Mett, I. L., Kochikyan, A. V. & Petrosyan, P. K. (1990). Molecular cloning and structural-functional analysis of arginine biosynthesis genes of the thermophilic bacterium Bacillus stearothermophilus. Genetika (USSR) 26, 1915-1925.
Sakanyan, V., Charlier, D., Legrain, C., Kochikyan, A., Mett, I., Piérard, A. & Glansdorff, N. (1993). Primary structure, partial purification and regulation of key enzymes of the acetyl cycle of aginine biosynthesis in Bacillus stearothermophilus: dual function of ornithine acetyltransferase. J. Gen. Microbiol. 139, 393-402.
Savchenko A., Weigel P., Dimova D., Lecocq M. & Sakanyan V. (1998). The Bacillus stearothermophilus argCJBD operon harbours a strong promoter as evaluated in Escherichia coli cells. Gene 212, 167-177. Studier, F. W., Rosenberg, A. H., Dunn, J. J. & Dubendorff, J. W. (1990). Use of 17 polymerase to direct expression of cloned genes. Methods Enzymol. 185, 60-89.
Thieffry, D., Salgado, H., Huerta, A. M. & Collado-Vides, J. (1998). Prediction of transcriptional regulatory sites in the complete genome sequence of Escherichia coli K-12. Bioinformatics 14, 391-400.
Thorson, J. S., Cornish, V. W., Barrett, J. E., Cload, S. T., Yano, T. & Schultz, P. G. (1998). A biosynthetic approach for the incorporation of unnatural amino acids into proteins. In: Methods Mol. Biol. vol. 77, Protein Synthesis: methods and protocols. Ed. R. Martin, Humana Press Inc., Totowa, N. J., p. 43-73.
Van Essen, A. J., Kneppers, A. L., van der Hout, A. H., Scheffer, H., Ginjaar, I. B., ten Kate, L. P., van Ommen, G. J., Buys, C. H. & Bakker, E. (1997). The clinical and molecular genetic approach to Duchenne and Becker muscular dystrophy: an updated protocol. J. Meth. Genet. 34, 805-812.
Zubay, G. (1973). In vim synthesis of protein in microbial systems. Ann. Rev. Genet. 7, 267-287.

1. A method for the identification of a nucleic acid sequence carrying a putative bacterial strong promoter, said method comprising: a. selecting among the sequences of a nucleic acid database, a putative promoter sequence of at least 50 nucleotides, preferably around 60-70 nucleotides, said putative promoter sequence being located upstream the initiation codon of an Open Reading Frame or a sequence corresponding to tRNA or rRNA, in a region which does not extend further than 500 nucleotides, preferably 300 nucleotides from said initiation codon, said putative promoter sequence comprising an UP element, said UP element consisting of either
the following consensus pattern: AAAWWTWTTTTNNNAAA (SEQ ID NO: 1), wherein “W” stands for any of the symbols “A” or “T” and “N” stands for any of the four symbols “A”, “T”, “G” or “C”; or,
a nucleotide sequence of the same length of SEQ ID NO:1 which can be aligned with SEQ ID NO:1 and having a score similarity sUP which is equal or superior to a minimum score similarity determined by the parameter scUP,
b. selecting among the sequences selected in step a., the sequences comprising a −35 site located from 0 to 5 nucleotides downstream the AT-rich UP element, said −35 site consisting of either
the following consensus pattern TCTTGACAT (SEQ ID NO 2), or
a nucleotide sequence of the same length of SEQ ID NO: 2 which can be aligned with SEQ ID NO: 2 and having a score similarity s35 which is equal or superior to a minimum score similarity parameter sc35; and
c. identifying among the sequences selected in step b., a sequence comprising a −10 site, downstream the −35 site, preferably at a distance of 14 to 20 nucleotides, preferably from 15 to 19, better from 16 to 18, and optimally 17 nucleotides from the −35 site, said −10 site consisting of either
the following consensus pattern TATAAT (SEQ ID NO: 3), or
a nucleotide sequence of the same length of SEQ ID NO: 3 which can be aligned with SEQ ID NO: 3 and having a score similarity s10 which is equal or superior to a minimum score similarity parameter sc10;
wherein sUP, s35 and s10 correspond to the sum of each coincidence rates of symbols in the corresponding alignments: the identity rate being equal to 1 and the non-identity rate being equal to 0.5 or 0 and determined for each pair compared of symbols as follows:
0.5 for pairs “A” to “T” or “T” to “A” and
0 for other possible pairs.

2. The method according to claim 1, wherein scUP is at least equal to 11, sc35 is at least equal to 5, and sc10 is at least equal to 4.

3. The method according to claim 1, wherein a normalised score tot_sc is attributed to each identified sequence according to the following equation:
tot_{—}sc=0.30*[1−(17−sUP)/20]+0.25*[1−(9−sc35)/10]+0.25*[1−(6−s10)²/10]+0.2*nsc_{—}dist, wherein nsc_dist is defined according to the following table:

| | | | | | |
|---|---|---|---|---|---|
| Distance between | 17 | 16, 18 | 15, 19 | 14, 20 | other |
| −35 site and −10 | | | | | |
| site in | | | | | |
| nucleotides | | | | | |
| Nsc_dist | 1 | 0.95 | 0.85 | 0.7 | 0.2 |

and the method further comprises the step of selecting the sequences having a normalised score tot_sc superior to 0.85.

4. The method according to claim 1, wherein said bacterial nucleic acid database comprise genomic sequence from bacteria which is used in industry and whose genome comprises a percentage of adenine and thymine inferior to 65%.

5. The method according to claim 1, wherein said bacterial nucleic acid database comprise genomic sequence from one bacterial specie selected from the group consisting of Thermotoga maritima, Mycobacterium tuberculosis, Mycobacterium leprae, Pseudomonas aeruginosa, Brucella melitensis, Neisseria meningitis, Salmonella typhimurium, Escherichia coli, Vibrio cholera, Yersinia. pestis, Streptococcus pneumoniae, Streptococcus pyogenes, Haemophilus influenzae and Helicobacter pylori.

6. The method according to claim 5, wherein said bacterial nucleic acid database comprises T. maritima genomic sequences.

7. A computer program comprising computer program code means for instructing a computer to perform the method of claim 1.

8. A computer readable storage medium having stored therein a computer program according to claim 7.

9. A method for the isolation of a nucleic acid having strong bacterial promoter activity, wherein said method further comprises the steps of:
a. isolating a nucleic acid having a putative strong bacterial promoter, said nucleic acid sequence being identified according to the method of claim 1,
b. determining promoter activity of the isolated nucleic acid as compared to a control bacterial strong promoter, such as the ptac promoter, wherein a higher promoter activity than the promoter activity of the control strong promoter indicates that said isolated nucleic acid has a strong bacterial promoter activity.

10. The method according to claim 2, wherein said bacterial nucleic acid database comprise genomic sequence from bacteria which is used in industry and whose genome comprises a percentage of adenine and thymine inferior to 65%.

11. The method according to claim 3, wherein said bacterial nucleic acid database comprise genomic sequence from bacteria which is used in industry and whose genome comprises a percentage of adenine and thymine inferior to 65%.

12. The method according to claim 2, wherein said bacterial nucleic acid database comprise genomic sequence from one bacterial specie selected from the group consisting of Thermotoga maritima, Mycobacterium tuberculosis, Mycobacterium leprae, Pseudomonas aeruginosa, Brucella melitensis, Neisseria meningitis, Salmonella typhimurium, Escherichia coli, Vibrio cholera, Yersinia. pestis, Streptococcus pneumoniae, Streptococcus pyogenes, Haemophilus influenzae and Helicobacter pylori.

13. The method according to claim 3, wherein said bacterial nucleic acid database comprise genomic sequence from one bacterial specie selected from the group consisting of Thermotoga maritima, Mycobacterium tuberculosis, Mycobacterium leprae, Pseudomonas aeruginosa, Brucella melitensis, Neisseria meningitis, Salmonella typhimurium, Escherichia coli, Vibrio cholera, Yersinia. pestis, Streptococcus pneumoniae, Streptococcus pyogenes, Haemophilus influenzae and Helicobacter pylori.


SEQUENCE LISTING (113 sequences)

SEQ ID NO: 1 - DNA, 17 bp, Artificial Sequence
  Promoter
aaawwtwttttnnnaaa

SEQ ID NO: 2 - DNA, 9 bp, Artificial Sequence
  Promoter
tcttgacat

SEQ ID NO: 3 - DNA, 6 bp, Artificial Sequence
  Promoter
tataat

SEQ ID NO: 4 - DNA, 115 bp, Thermotoga maritima
cggtttgtctttgagacgaatttaacagttttcttctgttcttagcgggtgatatttcaacattaaaatcttgacattctaccatgtcaaggtgtataatgcaaaatgtgaaaat

SEQ ID NO: 5 - DNA, 105 bp, Thermotoga maritima
atattcgatttccctcatatttaggtgcatgtatgtttttacaaattctcatacgaccccttgacatcccattctgtgcctcactataatttttcatggatgaga

SEQ ID NO: 6 - DNA, 142 bp, Thermotoga maritima
ggggttgtaagcaaaaggaaaactaatataatcaataataatcaaccatatttatctcttatagttcgatattaggattattttatactgaaagcccttgaccttgttgtatgtttgttgatattatattgataacttcaag

SEQ ID NO: 7 - DNA, 131 bp, Thermotoga maritima
gaaagaagacgtggaaagatttaaaatttaacagaaaataacaacttccacataagatgaaactgcattgtgatttttgtaactatattgacataaaacaaaaggtttgttataattcacatcgaggcata

SEQ ID NO: 8 - DNA, 123 bp, Thermotoga maritima
cctctctgagctcttctattctttttgtgatctccattgaaaacacctcccagattcaagtatatcctaaaaaaatatttgaaatgataccccaagattttatataattgattgatagaaaaa

SEQ ID NO: 9 - DNA, 111 bp, Thermotoga maritima
ctcataaaatccacctcccgggttaaaaattgttaaatatagattttcacattttgcattatacaccttgacatggtagaatgtcaagattttaatgttgaaatatcaccc

SEQ ID NO: 10 - DNA, 119 bp, Thermotoga maritima
gatattcataaacacgaaaataatatgtggatttcatcctttacaaaactgaaaataacagtgaaaaaacacttcatataaatcatttcaaataatcctataattatgctatcaagaac

SEQ ID NO: 11 - DNA, 110 bp, Thermotoga maritima
gagagttggaaagaggaagttctacagaatatcaggtggagagacaaaaaaactttagaaaactcttgaatttcctttggacgggatggtataatcaccacaggatttaa

SEQ ID NO: 12 - DNA, 111 bp, Thermotoga maritima
gccaggataaagaccattctcagagagagggagttaggcataaggaggtgaaaatatgcccaggaaacgtttgactggaatagttgtgagcgataaaatggacaagacagt

SEQ ID NO: 13 - DNA, 119 bp, Thermotoga maritima
catctttgcacttttcgttttcgccgtgggggtatggaaatatttcagaatgaaaaagaaggaagaaaaatgaaaacttgaacaaggaaacgattgagtgtataatatttttctggtgt

SEQ ID NO: 14 - DNA, 111 bp, Thermotoga maritima
taccaaggtacgtggtgaataaagaagtgatccgaattttgaaagaaaagggttatcaggaaatatcttgaatagaaaaggttcgtgtgttaaaataaccacacatacggg

SEQ ID NO: 15 - DNA, 116 bp, Thermotoga maritima
gctccttggaaagagcatcgggaataaaatcagttgtaactcaaagaaaatattagaatttgaactataattcgaaataattcctgttattcactcataatcataaaaaatgagta

SEQ ID NO: 16 - DNA, 103 bp, Thermotoga maritima
ccatatcgttacctattgtatcattttggaaacaaaaataaaaatttcatgaaaaatttcttgaattctgtgaccaaaagggtttaatatatagccatacggg

SEQ ID NO: 17 - DNA, 41 bp, B. Stearothermophilus
ccatggctatagaatttgtgataccaaaaaaattgaaggtg

SEQ ID NO: 18 - DNA, 27 bp, B. Stearothermophilus
gtcgacttcccccttcctgagctcaag

SEQ ID NO: 19 - DNA, 23 bp, B. Stearothermophilus
ggagggggaacatatgatgaagc

SEQ ID NO: 20 - DNA, 20 bp, B. Stearothermophilus
ggaccaccgcgctactgccg

SEQ ID NO: 21 - DNA, 53 bp, Artificial Sequence
  Promoter
gnaaaaatwtnttnaaaaaamncttgamannnnnnnnnnnnnnnnnntataat

SEQ ID NO: 22 - DNA, 8 bp, E. coli
gctggtgg

SEQ ID NO: 23 - DNA, 18 bp, Artificial Sequence
  Primer
gcgccgacatcataacgg

SEQ ID NO: 24 - DNA, 38 bp, Artificial Sequence
  Primer
catatgttccccctcctcacaattccacacattatacc

SEQ ID NO: 25 - DNA, 20 bp, Artificial Sequence
  Primer
gctccttggaaagagcatcg

SEQ ID NO: 26 - DNA, 36 bp, Artificial Sequence
  Primer
catatgttccccctcctactcattttttattatgag

SEQ ID NO: 27 - DNA, 25 bp, Artificial Sequence
  Primer
atattcgatttccctcatatttagg

SEQ ID NO: 28 - DNA, 38 bp, Artificial Sequence
  Primer
catatgttccccctcctctcatccatgaaaaattatag

SEQ ID NO: 29 - DNA, 19 bp, Artificial Sequence
  Primer
gagagttggaaagaggaag

SEQ ID NO: 30 - DNA, 36 bp, Artificial Sequence
  Primer
catatgttccccctccttaaatcctgtggtgattat

SEQ ID NO: 31 - DNA, 19 bp, Artificial Sequence
  Primer
ccatatcgtttacctattg

SEQ ID NO: 32 - DNA, 46 bp, Artificial Sequence
  Primer
catatgttccccctcccccgtatggctatatattaaacccttttgg

SEQ ID NO: 33 - DNA, 18 bp, Artificial Sequence
  Primer
ggggttgtaagcaaaagg

SEQ ID NO: 34 - DNA, 39 bp, Artificial Sequence
  Primer
catatgttccccctcccttgaagttatcaatataatatc

SEQ ID NO: 35 - DNA, 21 bp, Artificial Sequence
  Primer
cggtttgtctttgagacgaat

SEQ ID NO: 36 - DNA, 38 bp, Artificial Sequence
  Primer
catatgttccccctccattttcacattttgcattatag

SEQ ID NO: 37 - DNA, 18 bp, Artificial Sequence
  Primer
cccgctctctttctcatt

SEQ ID NO: 38 - DNA, 38 bp, Artificial Sequence
  Primer
catatgttccccctccattaaaatcttgacattctacc

SEQ ID NO: 39 - DNA, 18 bp, Artificial Sequence
  Primer
gaaagaagacgtggaaag

SEQ ID NO: 40 - DNA, 39 bp, Artificial Sequence
  Primer
catatgttccccctcctatgcctcgatgtgaattataac

SEQ ID NO: 41 - DNA, 19 bp, Artificial Sequence
  Primer
gccaggataaagaccattc

SEQ ID NO: 42 - DNA, 36 bp, Artificial Sequence
  Primer
catatgttccccctccactgtcttgtccattttatc

SEQ ID NO: 43 - DNA, 18 bp, Artificial Sequence
  Primer
cctctctgagctcttcta

SEQ ID NO: 44 - DNA, 33 bp, Artificial Sequence
  Primer
catatgttccccctcctttttctatcaatcaat

SEQ ID NO: 45 - DNA, 18 bp, Artificial Sequence
  Primer
gatattcataaacacgaa

SEQ ID NO: 46 - DNA, 39 bp, Artificial Sequence
  Primer
catatgttccccctccgttcttgatagcataattatagg

SEQ ID NO: 47 - DNA, 17 bp, Artificial Sequence
  Primer
catctttgcacttttcg

SEQ ID NO: 48 - DNA, 38 bp, Artificial Sequence
  Primer
catatgttccccctccacaccagaaaaatattatacac

SEQ ID NO: 49 - DNA, 18 bp, Artificial Sequence
  Primer
taccaaggtacgtggtga

SEQ ID NO: 50 - DNA, 54 bp, Artificial Sequence
  Primer
catatgttccccctcccccgtatgtgcccgtatgtgtggttattttaacacacg

SEQ ID NO: 51 - DNA, 46 bp, Thermotoga maritima
acaatttttatctgatattttcacattcaccatagtcgattataac

SEQ ID NO: 52 - DNA, 47 bp, Thermotoga maritima
atttattgtttaagtatttatgaaatgcattatgtatctgatacaat

SEQ ID NO: 53 - DNA, 51 bp, Thermotoga maritima
agaatgtgtgtaacaaaccatcgaaatcataagttattgactccatgtaat

SEQ ID NO: 54 - DNA, 49 bp, Thermotoga maritima
gacaatatattagaaattattgaaagcatccatgtgatgatgatacaat

SEQ ID NO: 55 - DNA, 45 bp, Thermotoga maritima
accttgattttaaattatcctgcatataattaatgtgaacataat

SEQ ID NO: 56 - DNA, 47 bp, Thermotoga maritima
acaatgtttgaatgatacttgaaatcagcgactgtgtgtagtacaat

SEQ ID NO: 57 - DNA, 50 bp, Thermotoga maritima
agatttatgttaactaaactataagataattctttgttgacagatatgat

SEQ ID NO: 58 - DNA, 49 bp, Thermotoga maritima
caatatttgtccagaaaacttgatttaacaaaaatggacaatgtagaat

SEQ ID NO: 59 - DNA, 51 bp, Thermotoga maritima
aaatgtgatatgaaaaatatggaacgataagttatcatatttctttttaat

SEQ ID NO: 60 - DNA, 49 bp, Thermotoga maritima genome
agaaaaatttttttggaacttgacaaaatatttggtaatattctaaaat

SEQ ID NO: 61 - DNA, 49 bp, Thermotoga maritima
ttttacaaattctcataccttgacatcccattctgtgcctcactataat

SEQ ID NO: 62 - DNA, 48 bp, Thermotoga maritima
tgtttttcttaatcaatccttgaagaggctcgtaaaaagtagtatatt

SEQ ID NO: 63 - DNA, 47 bp, Thermotoga maritima
taatgtaactattcaaatcattacagtttataattatgtggtaaaat

SEQ ID NO: 64 - DNA, 50 bp, Thermotoga maritima
gaatactctgtcagaaatcgtgatcatcttttcacctcgtgtagtataat

SEQ ID NO: 65 - DNA, 51 bp, Thermotoga maritima
attttaatgttaaattttaacgacatgggtggtaaaatctttccagataat

SEQ ID NO: 66 - DNA, 47 bp, Thermotoga maritima
caattttttcatatcattcttatagtggcactgctgaactctatatt

SEQ ID NO: 67 - DNA, 46 bp, Thermotoga maritima
aaaagaattgccagaaatcatgttatctcccccctccagttataaa

SEQ ID NO: 68 - DNA, 47 bp, Thermotoga maritima
aaaaatttcatgaaaaatcttgaattctgtgaccaaaagggtttaat

SEQ ID NO: 69 - DNA, 47 bp, Thermotoga maritima
tacaatattttgactgctcttgtagtcctctctgtttgttttataat

SEQ ID NO: 70 - DNA, 49 bp, Thermotoga maritima
gaaaagttacagaaaaaccttgttatctgaaggtgaaaaatggtaaaat

SEQ ID NO: 71 - DNA, 49 bp, Thermotoga maritima
tcattcattttaccatccacttgaaattcaggaaggtatgtagtacaat

SEQ ID NO: 72 - DNA, 48 bp, Thermotoga maritima
ttttttatctctactaaggttgacattattgattcagaagagtaaaat

SEQ ID NO: 73 - DNA, 47 bp, Thermotoga maritima
aaatatagattttcacatttgcattatacaccttgacatggtagaat

SEQ ID NO: 74 - DNA, 47 bp, Thermotoga maritima
agaaacaattttggaatccatggacattattacctttaatggataat

SEQ ID NO: 75 - DNA, 48 bp, Thermotoga maritima
aagaattctcttacaatcctggaatgtttccctcacagagaagataat

SEQ ID NO: 76 - DNA, 48 bp, Thermotoga maritima; identical to SEQ ID NO: 75

SEQ ID NO: 77 - DNA, 48 bp, Thermotoga maritima
agaaaaatttccgatgaacttgaaaagggtgaaaacctgtgctattat

SEQ ID NO: 78 - DNA, 48 bp, Thermotoga maritima
attgtgatttttgtaactattgacataaaacaaaaggtttgttataat

SEQ ID NO: 79 - DNA, 47 bp, Thermotoga maritima
atcttttcttagcgaagactggacgaaatggacaaattggctataat

SEQ ID NO: 80 - DNA, 48 bp, Thermotoga maritima
aaaaataaaaagtccttgattgaccatatttcgtactcatgctataat

SEQ ID NO: 81 - DNA, 48 bp, Thermotoga maritima
aagtatatcctaaaaaatttgaaatgataccccaagattttatataat

SEQ ID NO: 82 - DNA, 48 bp, Thermotoga maritima
tatgatactctgagaaacctggaataaagatcttttagaagctttaat

SEQ ID NO: 83 - DNA, 51 bp, Thermotoga maritima
aaaataacagtgaaaaaacttcatataaatcatttcaaataatcctataat

SEQ ID NO: 84 - DNA, 50 bp, Thermotoga maritima
aatattagaatttgaacaattcgaaataattcctgttattcactcataat

SEQ ID NO: 85 - DNA, 46 bp, Thermotoga maritima
aaaaatgtaaaagaagaacttgaatctttgaaaaacatcatatact

SEQ ID NO: 86 - DNA, 47 bp, Thermotoga maritima
aattcaaattacgtataatttgaattcacacataattattacataat

SEQ ID NO: 87 - DNA, 48 bp, Thermotoga maritima
aaatcatttttcttacctctggaaaagctttaaataaagtgttaaaat

SEQ ID NO: 88 - DNA, 48 bp, Thermotoga maritima
cttttcatttcaaaaaatttaacactttcgcagaaaaattggtagaat

SEQ ID NO: 89 - DNA, 46 bp, Thermotoga maritima
aaaataacagttcaacatattaacacacttcgccttgaagtataat

SEQ ID NO: 90 - DNA, 47 bp, Thermotoga maritima
aacaattctttagatgttctggatacattttgattagtttcaataat

SEQ ID NO: 91 - DNA, 46 bp, Thermotoga maritima
aaatatgtttgttgactttttaagattaattctctatcaatatgat

SEQ ID NO: 92 - DNA, 49 bp, Thermotoga maritima
ataaaaatttttctatatcgtgaaaaatttaacaattaaggttgataat

SEQ ID NO: 93 - DNA, 49 bp, Thermotoga maritima
aaaaaaactttagaaaatcttgaatttcctttggacgggatggtataat

SEQ ID NO: 94 - DNA, 51 bp, Thermotoga maritima
tgatattcgttctgaatttttacatttcatccaaattattttggttatagt

SEQ ID NO: 95 - DNA, 47 bp, Thermotoga maritima
aacttaagtaacacaaaccttgacaacgaaaggggggtgggtataat

SEQ ID NO: 96 - DNA, 50 bp, Thermotoga maritima
agaaattctttgaaaactctagaattcaaacgtcgcttttccagtatact

SEQ ID NO: 97 - DNA, 48 bp, Thermotoga maritima
tcaattattttaccaaaggttcaccatacgaactatttgttgtagaat

SEQ ID NO: 98 - DNA, 51 bp, Thermotoga maritima
ttctatattatgaaaatttcatggatattatccaaaaaattcatttatcat

SEQ ID NO: 99 - DNA, 48 bp, Thermotoga maritima
aaatataaatctgaattaattcacatttagcaaatcatcatttataat

SEQ ID NO: 100 - DNA, 49 bp, Thermotoga maritima
aaactgcttttaaaaaagattacattccgcagtaaacagaatttattat

SEQ ID NO: 101 - DNA, 51 bp, Thermotoga maritima
cgataatttttgcaatttctatacatctcacatcacctccggctatatatt

SEQ ID NO: 102 - DNA, 49 bp, Thermotoga maritima
attattttatactgaaaccttgaccttgttgtatgtttgttgatattat

SEQ ID NO: 103 - DNA, 49 bp, Thermotoga maritima
tgatatttcaacattaatcttgacattctaccatgtcaaggtgtataat

SEQ ID NO: 104 - DNA, 52 bp, Thermotoga maritima
aaaatgtttatgcaaatttctgttaaccatgttacacacaacatgtggtatc

SEQ ID NO: 105 - DNA, 47 bp, Thermotoga maritima
tcatattttgtgtaatttcctaacgttacacagcagtgtgataaaat

SEQ ID NO: 106 - DNA, 51 bp, Thermotoga maritima
aagttttgatttttgtaggttgaaataatctttctgacgatgtggtataat

SEQ ID NO: 107 - DNA, 48 bp, Thermotoga maritima
atatggaagttcaaaaatcttgctttcagagtgtgtttgtggtataaa

SEQ ID NO: 108 - DNA, 52 bp, Thermotoga maritima
agaaaactattggtaaaacttgaaatatatgactgtaaaaacgtgatataat

SEQ ID NO: 109 - DNA, 46 bp, Thermotoga maritima
tagtattctaccctaaatctttcattctggattcgataattgtaat

SEQ ID NO: 110 - DNA, 49 bp, Thermotoga maritima
aaaaagaaggaagaaaaaacttgaacaaggaaacgattgagtgtataat

SEQ ID NO: 111 - DNA, 48 bp, Thermotoga maritima
gtattattcattctaaaacttgaaactgaccaaataaagtattagaat

SEQ ID NO: 112 - DNA, 51 bp, Thermotoga maritima
taaaatccttagtgaaatttgtgaattttctgacggtaaactctttgtaat

SEQ ID NO: 113 - DNA, 47 bp, Thermotoga maritima
aaacgattcttctaaaatcttgatttgtatcactgttatgttataaa